(19) United States
(12) Patent Application Publication
(10) Pub. No.: US 2006/0204554 A1
(43) Pub. Date: Sep. 14, 2006

(54) Compositions and methods providing rumen bypass protein in ruminant diets

(76) Inventors: Michael J. Cecava, Decatur, IN (US); Perry H. Doane, Decatur, IN (US); James L. Dunn, Decatur, IN (US)

Correspondence Address: KIRKPATRICK & LOCKHART NICHOLSON GRAHAM LLP;HENRY W. OLIVER BUILDING, 535 SMITHFIELD STREET, PITTSBURGH, PA, 15222, US

(21) Appl. No.: 11/373,681

(22) Filed: Mar. 10, 2006

Related U.S. Application Data
(60) Provisional application No. 60/660,952, filed on Mar. 11, 2005.

(51) Int. Cl. A61K 36/06; A23K 1/165

(52) U.S. Cl. 424/442; 424/195.16; 426/635

(57) ABSTRACT

The present invention is based on the discovery that moist heat treated ruminant animal feed compositions comprising a fermentation biomass, have increased amounts of proteinaceous matter that escapes fermentation within the rumen. The ruminant animal feed compositions may further comprise, alone or in combination, one or more of an isolated enzyme, an organic acid, a gluten protein, at least one divalent metal ion and at least one plant extract. The proteinaceous matter may then be digested or metabolized in the post-rumen portions of the ruminant digestive system, thereby providing further increased energy and protein levels for ruminant animals during times of increased productivity. Compositions and methods of manufacture of the compositions of the embodiments of the present disclosure are disclosed.

### CROSS REFERENCE TO RELATED APPLICATIONS

This application claims priority to U.S. Provisional Application No. 60/660,952 filed Mar. 11, 2005, the disclosure of which is incorporated in its entirety by reference herein.

### BACKGROUND

1. Field of the Invention

The present invention relates to dietary compositions and methods for increasing production in a ruminant animal by slowing rumen fermentation of protein and thereby increasing post-rumen availability of protein and amino acids to the ruminant animal.

2. Background

Ruminant species are able to effectively utilize dietary ingredients that are poorly used by monogastric species. This occurs because ruminants can ferment dietary ingredients in the reticulo-rumen compartment of their complex ruminant stomach. Digestion of protein in the rumen has long been recognized as an important factor in the productive efficiency of ruminant diet formulation.

Ruminants meet their energy and protein requirements by a combination of rumen fermentation and digestion of protein that has escaped rumen fermentation. The production of protein and energy by rumen fermentation versus rumen escape followed by intestinal digestion and absorption varies widely among feedstuffs. The feed value of a dietary ingredient can also vary with animal productivity levels and/or animal diet formulation or composition.

As animal productivity levels increase, so do the nutritional requirements for amino acids, metabolizable protein and energy. At low productivity levels, nutritional requirements are more readily satisfied by rumen fermentation products. At elevated productivity levels, the gross efficiency of rumen nutrient digestion decreases. At such times, protein synthesis by rumen fermentation may not meet the animal's demands for metabolizable protein. This shortfall of rumen protein production increases the demand for rumen bypass protein. As defined herein, the terms “rumen bypass protein”, “rumen undegraded protein”, “rumen undegradable protein,” and “rumen escape protein”, mean proteins, peptides, and amino acid residues which escape fermentation in the rumen and pass, at least partially intact, into the post-rumen part of the digestive system. The bypass protein may then be metabolized by the post-rumen portions of the ruminant digestive system.

Research on increased productivity levels in ruminants has focused on the quantity and the quality of nutrients that escape rumen fermentation. The rumen escape of protein may be accomplished by processing dietary ingredients, thereby altering the physical structure of the protein therein and decreasing rumen fermentation, or by influencing rumen conditions so that the rumen bypass protein content of all dietary ingredients is increased.

A variety of methods have been used to reduce the rumen availability of vegetable protein. For example, U.S. Pat. No. 3,619,200 proposes a rumen-inert coating of vegetable meal for protection against rumen microbial digestion. Treatment of feeds with tannin, formaldehyde, or other aldehydes can denature the protein and reduce ruminal fermentation (see U.S. Pat. No. 4,186,213) and rumen digestion of protein can be reduced by heating (Tagari et al., Brit. J. Nutr. 16:237-243 (1982)).

Hudson presented an experiment evaluating the effect of heating soybean meal (“SBM”) on post-ruminal nitrogen utilization by lambs. The results indicated slower protein digestion by rumen microflora (Hudson et al., J. Anim. Sci. 30:609 (1970)). U.S. Pat. No. 5,508,058 to Endres et al., and U.S. Pat. No. 5,824,355 to Heitritter summarize the procedures commonly used for production of heat-treated vegetable meals.

Woodroofe et al. references pretreating a protein source with an enzyme prior to a process utilizing shear force, heat, pressure, and mixing to increase the amount of undigested protein passing through the rumen (U.S. Pat. No. 6,221,380). However, the approach requires shear force, heat and pressure to protect the protein from rumen fermentation.

The use of zinc metal salts to protect animal feed protein from rumen degradation has been disclosed by Meyer and Endres et al. in U.S. Pat. Nos. 4,664,905, 4,664,917, 4,704,287, 4,737,365, and 5,508,058. The use of manganese and iron with zinc have been shown to have a synergistic effect to improve bypass protein and animal performance in U.S. patent application Ser. No.10/246,720 to Cecava et al. (Publication No. 2003/0138524 A1).

As production levels in ruminant animals continue to increase, there are also increased requirements for metabolizable protein and amino acids. While dietary formulations increasing rumen bypass protein content in animal feed stuffs exist, there remains a demand for further improved animal feeds that provide further increased levels of protein that escapes rumen fermentation.

### SUMMARY

The present disclosure is directed toward improved animal feed compositions which increase the amount of proteinaceous matter that passes through the rumen of a ruminant animal, thereby increasing the amount of proteinaceous matter available for post-rumen digestion. Methods of making animal feed compositions according to the various non-limiting embodiments set forth herein are disclosed. Various methods for bypassing rumen protein digestion and increasing production in a ruminant animal are also disclosed.

One embodiment includes an animal feed composition comprising: at least one of an isolated enzyme, an organic acid, and a fermentation biomass of a eukaryotic cell origin and combinations of any thereof; and at least one proteinaceous feed ingredient, wherein the ingredient and the at least one proteinaceous feed ingredient are treated with a moist heat treatment, and wherein upon administration of the animal feed composition to a ruminant, an amount of protein passing through a rumen of the ruminant is increased as compared to an animal feed composition that does not include the treated ingredient and at least one proteinaceous feed ingredient administered to the ruminant.

Further embodiments include methods of feeding an animal. The method may comprise: treating a fermentation biomass of a eukaryotic cell origin and at least one proteinaceous feed ingredient; and feeding a ruminant an animal feed composition comprising the treated fermentation biomass and the at least one proteinaceous feed ingredient, wherein an amount of protein passing through a rumen of the ruminant is increased, upon administration of the animal feed composition to the ruminant, as compared to an animal feed composition that does not include the treated fermentation biomass and the treated at least one proteinaceous feed ingredient administered to the ruminant.

Other embodiments include a process for producing a feed supplement. The process comprises: mixing a composition comprising a fermentation biomass of a eukaryotic cell origin and at least one proteinaceous feed ingredient; treating the composition with moist heat; and forming the composition into a form selected from the group consisting of a meal, a pellet, a block, a tub, a premix, an additive, and a liquid feed supplement.

Still another embodiment includes an animal feed composition comprising: a yeast fermentation biomass; and at least one proteinaceous feed ingredient, wherein the yeast fermentation biomass and the at least one proteinaceous feed ingredient have been treated. Upon administration of the animal feed compositions according to this embodiment to a ruminant, an amount of protein passing through a rumen of the ruminant is increased as compared to an animal feed composition that does not comprise the yeast fermentation biomass.

### BRIEF DESCRIPTION OF THE FIGURES

FIGS. 1-3 are graphs of the percent protein recovered for moist heat treated SBM combined with the enzyme alpha-galactosidase or xylanase over 72 hours of rumen fermentation;

FIG. 4 shows the effect on percent content of rumen undegraded protein (“RUP”) from varying amounts of ascorbic acid in moist heat treated SBM;

FIG. 5 shows the effect on percent content of RUP from varying amounts of citric acid in moist heat treated SBM;

FIG. 6 shows the effect on percent content of RUP from varying amounts of ascorbic acid in moist heat treated SBM heated for either 4 or 5 hours;

FIG. 7 shows the effect on percent content of RUP from increasing divalent metal ion concentrations in moist heat treated SBM combined with 0.5% ascorbic acid (w/w);

FIG. 8 shows the effect on percent content of RUP from increasing ascorbic acid concentrations in moist heat treated SBM combined with 1500 ppm of a mixture of divalent metal ions; and

FIG. 9 shows the effect on percent content of RUP from increasing divalent metal ion concentrations in moist heat treated SBM combined with 1% citric acid (w/w).

### DETAILED DESCRIPTION OF THE EMBODIMENTS

The present invention is based on the discovery that moist heat treated ruminant animal feed compositions comprising proteinaceous feedstuffs, a fermentation biomass of a eukaryotic origin and, optionally, one or more of a gluten protein, an isolated enzyme, and an organic acid, which may be used alone or in combination, have increased amounts of proteinaceous matter that escapes fermentation within the rumen. The ruminant animal feed compositions may further comprise, alone or in combination, proteinaceous feedstuff, a fermentation biomass, and, optionally, one or more of a gluten protein, an isolated enzyme, an organic acid, and at least one divalent metal ion and/or at least one plant extract. As used herein, the term “proteinaceous feedstuffs” means any material comprising proteins that may be fed to a ruminant animal. Examples of suitable proteinaceous feedstuffs include, but are not limited to, soybean meal, corn meal, linseed meal, cottonseed meal, canola meal, and the meal of any grain edible to ruminants.

The proteinaceous matter may then be digested or metabolized in the post-rumen portions of the ruminant digestive system, thereby providing further increased energy and protein levels for ruminant animals during times of increased productivity. Compositions and methods of manufacture of the compositions of the embodiments of the present disclosure are disclosed. In addition, methods of bypassing rumen protein digestion and increasing production of a ruminant animal, comprising feeding the animal the compositions of the embodiments of the present disclosure, are also disclosed.

Other than in the operating examples, or where otherwise indicated, all numbers recited herein expressing quantities of ingredients, reaction conditions and the like are to be understood as being modified in all instances by the term “about”. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical values, however, inherently contain certain errors necessarily resulting from the standard deviation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

Any patent, publication, or other disclosure material, in whole or in part, that is identified herein is incorporated by reference herein but only to the extent that the incorporated material does not conflict with existing definitions, statements, or other disclosure material set forth in this disclosure. As such, and to the extent necessary, the disclosure as set forth herein supersedes any conflicting material incorporated herein by reference. Any material, or portion thereof, that is incorporated by reference herein, but which conflicts with existing definitions, statements, or other disclosure material set forth herein will only be incorporated to the extent that no conflict arises between that incorporated material and the existing disclosure material.

In certain embodiments of the present disclosure, composition may be processed or treated with moist heat to increase the content of rumen undegraded protein in ruminant feed compositions when compared to ruminant feed compositions that have not been processed or treated with moist heat. As used herein “moist heat” treatments suitable for the methods and compositions of the present disclosure may include, without limitation, heating a composition under conditions of 10% to 50% moisture to a temperature of 88° C. to 116° C. for 0.10 to 5 hours. For example, one moist heat treatment method suitable for use in the present disclosure may include the AminoPLUS® processing conditions (registered trademark of Ag Processing Inc. of Omaha, Nev.) (summarized in U.S. Pat. No. 5,824,355 to Heitritter et al., the disclosure of which is incorporated herein by reference) or modifications thereof, such as, for example, a moist low-heat pre-treatment. One processing method modification that is suitable for use in certain non-limiting embodiments of the methods and compositions of the present disclosure comprises: pre-treating the composition at a moisture level of 25% to 50% moisture for 0.10 hours to 5 hours at a temperature from 20° C. to 45° C. Pre-treatment processing methods may be a desirable modification when the composition contains one or more of an enzyme. As used herein, the terms “moist heat treated” and “treated with moist heat” are defined as treating or processing the feed composition with conditions as described above.

Compositions within the various non-limiting embodiments of the present disclosure may comprise at least one ingredient selected from the group consisting of an isolated enzyme, an organic acid, and a fermentation biomass of a eukaryotic cell origin. Certain non-limiting embodiments of the compositions comprise an isolated enzyme. As used herein, the term “isolated enzyme” is defined as an isolated compound comprised of at least one protein chain that is capable of catalyzing or increasing the rate of a biochemical reaction or process. The at least one ingredient according to the various non-limiting embodiments of the present disclosure may be used independent of or in combination with further ingredients of the present disclosure, as discussed below, to increase the content of rumen undegraded protein in ruminant feed compositions, such as, for example, soybean meal, when compared to rumen undegraded protein content of ruminant feed compositions without the isolated enzyme and/or further ingredients. Without intending to be limited to a particular mechanism for the embodiments of the present disclosure, it is believed that the ingredient may affect the reaction of proteins with sugars via a Maillard-type reaction, thereby slowing the digestion of protein in the rumen and increasing the quantity of protein matter that passes at least partially intact into the post-rumen portions of the ruminant digestive system.

The Maillard reaction, also known as non-enzymatic browning, involves the thermal reaction between an aldose or a ketose and alpha-amino acids or amino acid residues in proteins to afford a resulting Schiff base. The Schiff base residues may undergo subsequent rearrangement to form a more stable structure known as the Amadori product. Further reaction may lead to the formation of indigestible melanoidins (D. W. S. Wong, Food Chemistry and Biochemistry, in Encyclopedia of Food Science and Technology, 2nd ed., F. J. Francis, ed., Wiley & Sons, 2000, vol. 2, pp 877-880). Utilization of the early stages of the Maillard reaction leads to amino acid or protein residues that are protected from fermentation within the rumen microflora environment and therefore tend to escape fermentation in the rumen to be metabolized in the post-rumen portions of the ruminant digestive system.

Isolated enzymes suitable for use in the various non-limiting embodiments of the present disclosure include, but are not limited to, alpha-galactosidase (available from Kemin Industries, Inc., Des Moines, Iowa), xylanase, including xylanase Thermomyces lanuginosus (temperature-resistant xylanase), and Xylanase Cocktail (a combination of xylanase, hemicellulase, cellulose, and alpha-galactosidase available from D.F. International, LLC, Gaithersburg, Md.) and mixtures thereof. Other isolated enzymes that may be suitable for use in various non-limiting embodiments of the composition include, but are not limited to, cellulase, protease, hemicellulase, alpha-amylase, beta-glucanase, and pectinase. In one non-limiting embodiment of the compositions of the present disclosure, the isolated enzyme is added to the composition in an amount of from 0.030 grams of enzyme/kilogram of composition (g/kg) (0.60 lbs/ton) to 2.2 g/kg (4.4 lbs/ton). In another non-limiting embodiment of the present disclosure, the isolated enzyme is added to the composition in an amount from 0.050 g/kg (0.10 lbs/ton) to 0.40 g/kg (0.80 lbs/ton). As will be appreciated by those skilled in the art, certain commercially available enzymes comprise a mixture of active and inactive enzyme, typically expressed in units of active enzyme vs. a standard substrate. Therefore, the concentrations of the isolated enzymes, expressed above, are based on the concentration of active enzyme found in the isolated enzyme composition being used. Selection of a suitable isolated enzyme for various non-limiting embodiments of the compositions disclosed herein may depend on the nature of the composition. For example, in certain non-limiting embodiments comprising an isolated enzyme, wherein the enzyme comprises xylanase, the concentration of active enzyme added to the compositions is from 0.005% to 0.04%, by weight. In other non-limiting embodiments wherein the isolated enzyme comprises alpha-galactosidase, the concentration of active enzyme added to the composition is from 0.003% to 0.22% by weight.

Compositions within certain non-limiting embodiments of the present disclosure may comprise an organic acid. As used herein, the term “organic acid” is defined as any member of the class of acidic organic molecules having from two to nine carbon atoms with at least one acidic oxygen-hydrogen bond. According to the various non-limiting embodiments disclosed herein, the organic acids may be used independently of or in combination with the isolated enzyme, as discussed above, and/or in combination with the fermentation biomass or further ingredients of the present disclosure, as discussed below, to increase the quantity of protein in ruminant feed compositions that escapes rumen fermentation when compared to ruminant feed compositions without the organic acids.

Organic acids suitable for use within specific non-limiting embodiments of the present disclosure include, but are not limited to, ascorbic acid, citric acid, aconitic acid, malic acid, fumaric acid, succinic acid, lactic acid, malonic acid, maleic acid, tartaric acid, aspartic acid, oxalic acid, tatronic acid, oxaloacetic acid, isomalic acid, pyrocitric acid, glutaric acid, ketoglutaric acid, and mixtures thereof. The organic acids according to certain non-limiting embodiments, may be added to the composition as the free-acid or as a salt. Suitable organic acid salts include, but are not limited to, sodium salts, potassium salts, magnesium salts, calcium salts, and ammonium salts. In one non-limiting embodiment, the organic acid or salt thereof, such as ascorbic acid, citric acid, aconitic acid, malic acid, fumaric acid, succinic acid, lactic acid, malonic acid, maleic acid, tartaric acid, aspartic acid, pyrocitric acid, or mixtures and salts thereof, may be added to the compositions of the present disclosure in amounts from 0.1% to 6.0% by weight. In another non-limiting embodiment of the compositions of the present disclosure, the organic acid may be added to the composition in amounts from 1% to 4% by weight. In another non-limiting embodiment, the organic acid may be added to the composition in amounts from 0. 5% to 1.5% by weight. Without intending to be limited to any particular theory, the organic acids may act as catalysts in the Maillard reaction of the protein and saccharide residues within the feed to form rumen indigestible Schiff-base compounds.

According to various non-limiting embodiments, the combination of organic acids with other components within certain compositions within the present disclosure may display additional increase in rumen bypass protein in ruminant feed compositions. For example, as discussed below, in certain non-limiting embodiments, the combinations of an organic acid with certain metal ions have shown a synergistic increase in the percent of protein bypassing rumen fermentation compared to the increase associated with either an organic acid or the metal ions alone. In one non-limiting embodiment where the composition further comprises at least one metal ion, as disclosed below, the organic acid may be added to the composition in amounts from 0.10% to 1.5% by weight.

Further non-limiting embodiments of the compositions of the present disclosure may comprise a fermentation biomass and mixtures of fermentation biomasses, such as, but not limited to, fermentation biomasses of eukaryotic origin. As used herein, the term “fermentation biomass” is defined as the by-products left over from an aqueous fermentation process, such as an ethanol, lactate, lysine, fungal, or bacterial fermentation. The biomass may comprise the mycelium of a yeast or fungal fermentation and the media on which it was grown and may comprise the enzyme system of the viable organism and its concomitant metabolites produced during the fermentation process and not removed during the separation process. The biomass may further or alternatively comprise a bacterial fermentation mass and the media on which it was grown and may comprise the enzyme system of the viable organism and its concomitant metabolites produced during the fermentation process and not removed during the separation process.

Suitable fermentation biomass sources for use in certain non-limiting embodiments of the present disclosure include, but are not limited to, ethanol presseakes, such as presscakes of brewer's yeast or baker's yeast (Saccharomyces cerevisiae), distiller's yeast biomasses, propagated yeast biomass, citric acid presscakes, biomasses from lactic acid fermentations, biomasses from bacterial fermentations, and biomasses from lysine fermentations and mixtures thereof. Yeast organisms suitable for use in the various non-limiting embodiments of the compositions disclosed herein may be any of a number of yeast including, but not limited to, the Saccharomyces, Candida, Pichia, Yarrowia, Kluyveromyces, or Torulaspora species. In certain non-limiting embodiments, the yeast used may be Pichia guilliermondii or Yarrowia Lipolytica.

As used herein, the term “yeast culture” is defined as the product comprising mycelium of yeast fermentation and the media on which it was grown, such as, for example, a presscake. The yeast culture comprises the enzyme system of the viable organism and its concomitant metabolites produced during the fermentation process and not removed during the separation process. The process of separation includes, but is not limited to, filtration and pressing, and centrifugation. The fermentation process can be, but is not limited to, a penicillium fermentation, a Streptomyces fermentation, an ethanol fermentation, or a citric acid fermentation.

As used herein, the term “presscake” means the filtered or centrifuged; and dried mycelium obtained from separation of the fermentation. The term “citric acid presscake”, as used herein, means the filtered or centrifuged; and dried mycelium obtained from a citric acid fermentation using an acceptable aqueous carbohydrate substrate. The term “ethanol presscake” is defined as the filtered or centrifuged mycelium obtained from an ethanol fermentation using an acceptable aqueous carbohydrate substrate. The yeast organism may be made nonviable and may be completely removed from the citric acid or ethanol during the separation and purification process. Citric acid presscakes can be a product resulting from Pichia or Yarrowia yeast fermentation to produce citric acid, in which case it contains cell walls and cell wall contents with high concentrations of mannanoligosaccharides, fructooligosaccharides, and/or beta-glucans. The oligosaccharides and yeast cultures that may be used in the compositions of the present disclosure may be obtained, for example, from a variety of commercial sources.

In the various non-limiting embodiments of the present disclosure comprising a biomass, the biomass may comprise from 0.50% up to 99%, by weight, of the composition. In certain non-limiting embodiments, the biomass dry matter may comprise from 0.25% to 5.00% of the composition by weight based on dry weight of the composition. In other non-limiting embodiments, the biomass may comprise from 0.50% to 2.30% of the composition by weight based on dry weight of the composition. In various non-limiting embodiments the biomass may be added to the composition as a wet biomass. According to the embodiments wherein a wet biomass is added to the composition, the wet biomass is added in quantities from 2.5% of the total added moisture to 35% of the total added moisture. According to various non-limiting embodiments, the total added moisture may vary from 10% total added moisture to 45% total added moisture, as described above with regard to the pre-treatment process for the moist heat treatment. In certain non-limiting embodiments, the total moisture may be from 10% to 25% and the wet biomass added in an amount from 2.5% to 25%.

As used herein, the term “presscake” is defined as the filtered or centrifuged mycelium obtained from separation of the fermentation. The term “citric acid presscake”, as used herein, is defined as the filtered or centrifuged mycelium obtained from a citric acid fermentation using an acceptable aqueous carbohydrate substrate. The term “ethanol presscake” is defined as the filtered or centrifuged mycelium obtained from an ethanol fermentation using an acceptable aqueous carbohydrate substrate. Without intending to be limited by any particular interpretation, it is believed that the fermentation biomass may be beneficial during moist heat processing as a useful source of residual organic acids, such as fatty acids, and reducing sugars.

In certain non-limiting embodiments, the compositions of the present disclosure may additionally comprise a gluten protein from a cereal grain. As used herein, the term “gluten protein” is defined as a storage protein classified in four types according to their solubility: albumins which are soluble in water or aqueous salt solutions, globulins which are insoluble in water but soluble in dilute salt solutions, prolamins which are soluble in alcohol, and glutelins which are soluble in dilute acid or base. The prolamins have been considered to be unique to the seeds of cereals and other grasses and unrelated to other proteins of seeds or other tissues. The prolamins have been given different names in different cereals, such as: gliadin in wheat, avenins in oats, zeins in maize, secalins in rye, and hordein in barley.

Suitable gluten proteins that may be incorporated into the compositions of various non-limiting embodiments of the present disclosure include, but are not limited to, wheat gluten proteins, corn gluten proteins, oat gluten proteins, rye gluten proteins, rice globulin proteins, barley gluten proteins, and mixtures thereof. In one non-limiting embodiment, the gluten protein comprises corn gluten. In another non-limiting embodiment, the gluten protein comprises wheat gluten. In another non-limiting embodiment, the gluten protein comprises rice globulin proteins. The gluten proteins of the compositions of the present disclosures may be added to the compositions in the form of the isolated gluten proteins, or as a gluten meal. In various embodiments of the present disclosure comprising a gluten protein, such as corn gluten protein, wheat gluten protein, or rice globulin proteins, the gluten protein may comprise from 0.25% to 50.0%, by weight, of the composition.

Gluten meals typically contain more protein and have a higher content of rumen bypass protein than soybean meal. In one non-limiting embodiment of the compositions of the present disclosure, the gluten meal is added to the composition in an amount from 0.25% to 50% by weight. In one non-limiting embodiment, the gluten meal is added to the composition in an amount from 0.25% to 20% by weight. In another non-limiting embodiment, the gluten meal is added to the composition in an amount from 10% to 50% by weight. According to certain non-limiting embodiments, compositions comprising mixtures of a protein blend and 10% to 50%, by weight, gluten meal, such as corn gluten meal and wheat gluten meal, when treated with moist heat, show significantly increased levels of rumen undegraded protein content relative to the weighted average of the rumen undegraded protein content of the protein blend and the gluten meal. Without intending to be limited to any particular mechanism, it is believed that the gluten proteins may associate with other proteins in the feed mixture as they are treated with moist heat. The gluten protein and the associated feed protein may become insoluble in the rumen environment and protected from fermentation within the rumen. One skilled in the art will recognize that the level of protection provided by the gluten meal may be dependent upon the processing conditions and the amount and type of gluten protein used in the process. When combined with other components of certain embodiments of the compositions of the present disclosure, gluten proteins, such as in the form of gluten meals, may show further increased levels of rumen undegraded protein content, compared to compositions that do not contain gluten proteins.

According to certain non-limiting embodiments of the present disclosure, certain non-gluten proteins that are highly responsive to formation of rumen undegraded protein, or non-gluten proteins that are naturally high in rumen undegraded protein content may be effectively associated with other proteins, such as feed blend proteins, in a mixture providing greater levels of rumen fermentation protection that would normally be expected from the weighted average values for rumen undegraded protein content. According to these non-limiting embodiments, non-gluten proteins that may also show increased values of rumen undegraded protein content when mixed with a feed blend protein include, but are not limited to, milk proteins, egg proteins, and blood products such as blood meal.

The use of divalent metal ions of zinc, manganese and iron has been shown to have a direct influence on the rumen fermentation of protein (see, U.S. patent application Ser. No. 10/246,720 to Cecava et al. (Publication No. 2003/0138524), the disclosure of which is incorporated in its entirety by reference herein). The addition of divalent metals to feed proteins may act by changing the protein structure or by altering the rumen environment, or both. Experiments have shown that the influence of divalent metal ions on the levels of rumen bypass protein content is further increased when the metal ions are incorporated into the compositions of the present disclosure. Therefore, certain non-limiting embodiments of the compositions of the present disclosure may further comprise one or more divalent metal ions. Non-limiting examples of metal ions suitable for use in various non-limiting embodiments of the compositions of the present disclosure are water soluble salts, for example, sulfate salts, of divalent zinc, divalent manganese and divalent iron, although it is important to note that all water soluble salts, and combinations of metals or metal salts, may be used in the practice of the present disclosure. According to certain non-limiting embodiments, metal salts may be added to the compositions of the present disclosure either as a single chemical entity or as a mixture of more than one salt composition, which may include salts containing the same metal ion and salts with differing metal ions.

In one non-limiting embodiment of certain compositions of the present disclosure comprising divalent metal salts, a combination of zinc-salts, manganese salts and ferrous iron salts are added to the composition in equal concentrations (as measured in parts per million (“ppm”)). Metal salts may be added to certain compositions of the present disclosure in a total amount from 225 ppm to 4,000 ppm of the zinc, manganese and ferrous iron salt combination (from 75 ppm to 1,333 ppm of each type of metal ion). In one non-limiting embodiment in which metal salts are included in the compositions, the metal salts may be added in a total amount of 600 ppm to 3,000 ppm of the zinc, manganese and ferrous iron salt combination (from 200 ppm to 1,000 ppm of each type of metal ion).

According to the various non-limiting embodiments disclosed herein comprising metals, the amount of metal salt or combination of metal salts will vary according to the presence and amount of other components within a specific embodiment of the composition. In one non-limiting embodiment, the composition may comprise from 0.1% to 1.0% by weight of an organic acid, such as, for example, ascorbic acid or citric acid, and from 600 ppm to 3,000 ppm of a mixture of equal amounts of metal ions, such as, for example, zinc, manganese and ferrous iron metal ion.

According to one non-limiting embodiment, for example, the combination of an organic acid, such as citric acid, with ferrous iron salt shows a significant increase in rumen bypass protein content when compared to the combination of the organic acid with either zinc or manganese salts or when compared to the effect of the organic acid or the ferrous iron salt alone. According to this non-limiting embodiment, the composition may comprise from 0.5% to 1.5% of an organic acid, such as, for example, citric acid, and one or more metal ions in an amount from 500 ppm to 1500 ppm, such as, for example, 500 ppm to 1500 ppm of ferrous iron metal ions.

In another non-limiting embodiment, for example, the combination of an isolated enzyme, such as xylanase, with a combination of equal amounts of zinc, manganese and ferrous iron salts, when the metal concentrations are from 1,000 ppm to 2,000 ppm each, shows increased content of rumen bypass protein, when compared to the increase in rumen bypass protein content from the addition of xylanase or zinc alone.

In another non-limiting embodiment, the combination of one or more metals, such as zinc, manganese and/or ferrous iron salts, with one or more plant extract shows increase the content of rumen bypass protein, when compared to the increase in rumen bypass protein from the addition of metal or plant extract alone.

Traditionally, plant extracts have been added to animal feeds as-flavoring agents. The addition of plant extracts is not generally known for the purpose of increasing the rumen bypass protein content of animal feeds. Accordingly, certain non-limiting embodiments of the compositions of present disclosure may further comprise at least one plant extract, wherein the at least one plant extract, either alone or in combination with one or more of the other ingredients of the composition, increases the rumen undegraded protein content of the composition when compared to a composition without the at least one plant extract. As used herein, the term “plant extract” is defined as a compound in any form, for example a liquid, an oil, a crystal, or a dry powder, isolated from a botanical source that can be incorporated into certain non-limiting embodiments of the compositions of the present disclosure. Plant extracts suitable for use in certain non-limiting embodiments of the present compositions include, but are not limited to, saponins from yucca plants, saponins from quillaja plants, saponins from soybeans, tannins, cinnamaldehyde, eugenol or other extracts of clove buds, including clove oil or clove powder, garlic extracts, cassia extracts, capsaicin, anethol or mixtures thereof.

In certain non-limiting embodiments of the present disclosure, the plant extract may be formed into a base mixture by mixing the extract with an oil, such as canola oil, for ease of concentration measurement and dispensing. According to various non-limiting embodiments, plant extracts may be added to the compositions of the present disclosure either as a single extract or a combination of two or more different extracts. According to one non-limiting embodiment, the plant extracts may comprise garlic oil may be combined with cassia oil. In another non-limiting embodiment, the plant extracts added to the composition may comprise the combination of eugenol and cinnamaldehyde. In any event, according to these non-limiting embodiments the plant extract may be added in an amount where the content of rumen undegraded protein in a ruminant feed composition is increased relative to a feed composition without the plant extract additive. For example, in one non-limiting embodiment, the one or more plant extract may be added in an amount from 50 ppm to 2,500 ppm.

In certain non-limiting embodiments of the compositions of the present disclosure, the composition may further comprise the combination of one or more metal ions and at least one plant extract. This combination may have a positive synergistic effect wherein the rumen undegraded protein content in a ruminant feed composition of the present disclosure comprising one or more metal ions and at least one plant extract is increased when compared to ruminant feed composition of the present disclosure comprising one or more metal ions and no plant extract or comprising at least one plant extract and no metal ions. For example, in one-non-limiting embodiment, the combination of divalent metal ions, such as zinc, and saponins of yucca show reduced production of ammonia in the rumen. Ammonia production may be associated with the microbial fermentation digestion of protein within the rumen. Thus, a reduction in ammonia production in the rumen may be associated with a reduction in the amount of protein digested in the rumen and an increase of the quantity of protein passing substantially intact into the post-rumen portions of the ruminant digestive system.

According to one non-limiting embodiment of the compositions of the present disclosure, the animal feed composition comprises an isolated enzyme, an organic acid, a fermentation biomass, a gluten protein, at least one divalent metal ion, and at least one plant extract. According to this non-limiting embodiment, the composition is treated with moist heat using one of the moist heat treatment methods disclosed herein, such that when a ruminant animal is fed the composition, the amount of protein nutrients passing through the rumen and into the latter parts of the ruminant digestive tract is increased, compared to when an animal is fed a composition that does not include one or more of the above components.

The present disclosure also contemplates various non-limiting methods of bypassing protein digestion in the rumen. One non-limiting embodiment of such a method contemplated by the present disclosure comprises feeding a ruminant a moist heat treated composition comprising at least one of an isolated enzyme, an organic acid and a fermentation biomass of a eukaryotic cell origin, as described herein and set forth in the claims. Another non-limiting embodiment of such a method contemplated by the present disclosure comprises feeding a ruminant a moist heat treated composition comprising an isolated enzyme; an organic acid; a fermentation biomass; a gluten protein; at least one divalent metal ion; and at least one plant extract, as described herein and set forth in the claims.

According to other embodiments, the present disclosure provides for an animal feed composition comprising: an ingredient selected from the group consisting of an isolated enzyme, an organic acid, a fermentation biomass of a eukaryotic cell origin, and combinations of any thereof; and at least one proteinaceous feed ingredient. The ingredient and the at least one proteinaceous feed ingredient may be treated with a moist heat treatment. Upon administration of the animal feed composition to a ruminant, the amount of protein passing through the rumen of the ruminant (i.e., rumen bypass protein) is increased, as compared to an animal feed composition that does not include the ingredient administered to the ruminant.

According to certain embodiments, the animal feed comprises a fermentation biomass of a eukaryotic cell origin. As used herein, fermentation biomasses of a eukaryotic cell origin includes fermentation biomasses from yeast and yeast fermentation origins. According to various embodiments, the fermentation biomass of a eukaryotic cell origin may be selected from the group consisting of a yeast, a yeast cream, a yeast biomass, a lysine biomass, a lactic acid fermentation biomass, a citric acid presscake, an ethanol presscake, distiller's yeast, a brewer's yeast biomass, a baker's yeast biomass, and mixtures of any thereof. For example, according to certain embodiments, the fermentation biomass may be a yeast selected from the group consisting of brewer's yeast biomass and baker's yeast biomass (both of which may be derived from Saccharomyces cerevisiae). According to certain embodiments, the fermentation biomass according to these methods may not be of a prokaryotic origin, for example, a bacterial fermentation biomass, such as solubles from a glutamic acid fermentation. According to various embodiments, the fermentation biomass may be added to the proteinaceous feed ingredient in an amount of 1% to 20% by weight of the moist animal feed composition. According to other embodiments, the fermentation biomass may be added in 5% to 15% by weight of the moist animal feed composition. According to other embodiments, the fermentation biomass is added in an amount of 7% to 8% by weight of the moist animal feed composition.

The animal feed comprising the ingredient and at least one proteinaceous feed ingredient may comprise a proteinaceous feed ingredient, such as, plant and vegetable proteins, including edible grains and grain meals selected from the group consisting of soybeans, soybean meal, corn, corn meal, linseed, linseed meal, cottonseed, cottonseed meal, rapeseed, rapeseed meal, sorghum protein, and canola meal. Other examples of proteinaceous feed ingredients may include; corn or a component of corn, such as, for example, corn fiber, corn hulls, silage, ground corn, or any other portion of a corn plant; soy or a component of soy, such as, for example, soy hulls, soy silage, ground soy, or any other portion of a soy plant; wheat or any component of wheat, such as, for example, wheat fiber, wheat hulls, wheat chaff, ground wheat, wheat germ, or any other portion of a wheat plant; canola or any other portion of a canola plant, such as, for example, canola protein, canola hulls, ground canola, or any other portion of a canola plant; sunflower or a component of a sunflower plant; sorghum or a component of a sorghum plant; sugar beet or a component of a sugar beet plant; cane sugar or a component of a sugarcane plant; barley or a component of a barley plant; corn steep liquor; a waste stream from an agricultural processing facility; soy molasses; flax; peanuts; peas; oats; grasses, such as orchard grass and fescue, and alfalfa, clover used for silage or hay, and various combinations of any of the feed ingredients set forth herein.

According to certain embodiments, the animal feed composition comprising the ingredient and the at least one proteinaceous feed ingredient may further comprise an organic acid. The organic acid is as described herein and may be selected from the group consisting of ascorbic acid, citric acid, aconitic acid, malic acid, fumaric acid, succinic acid, pyrocitric acid, lysine, salts of any thereof, and combinations of any thereof. According to other embodiments, the animal feed composition comprising the ingredient and the at least one proteinaceous feed ingredient may further comprise a gluten protein, such as the gluten proteins described herein. For example, the gluten protein may be one of a corn gluten protein, a rice globulin protein, a wheat gluten protein, and mixtures of any thereof. According to still other embodiments, the animal feed composition may further comprise one or more ingredients selected from the group consisting of an isolated enzyme, a divalent metal ion, and a plant extract, as described herein.

The animal feed composition comprising the ingredient and the at least one proteinaceous feed ingredient, as described herein, may be treated with a moist heat treatment. The moist heat treatment may be as described herein and may comprise mixing a 50:50 mixture of the ingredient and water; and combining sufficient amounts of the aqueous ingredient mixture with the proteinaceous feed ingredient(s) to provide a moisture level of 15% to 50% added water (i.e., moisture content). In certain embodiments, the aqueous ingredient mixture may be added in sufficient amount to provide a 15% to 25% added water. In other embodiments, the aqueous ingredient mixture may be added in sufficient amount to provide a 15% added water. According to certain embodiments, the ingredient comprises a fermentation biomass of a eukaryotic cell origin.

Treating with moist heat may further comprise heating the animal feed composition comprising the ingredient and at least one proteinaceous feed ingredient at a temperature from 87° C. to 116° C. with 15% to 50% moisture for a time of 0.10 hours to 5 hours and then drying the composition to 10% to 15% moisture. According to certain embodiments, the composition may be dried to approximately 12% moisture, for example, in an oven at a temperature of 50° C. During the moist heat treatment the animal feed composition is heated in a system such that at least a substantial portion of the moisture is not lost, for example by heating in a substantially covered container.

According to other embodiments, the present disclosure also includes methods for feeding an animal. According to certain embodiments, the methods may comprise: treating a fermentation biomass of a eukaryotic cell origin and at least one proteinaceous feed ingredient; and feeding a ruminant an animal feed composition comprising the treated fermentation biomass and the at least one proteinaceous feed ingredient. According to these methods, the amount of protein passing through the rumen (i.e., rumen bypass protein) of the ruminant may be increased upon administration of the animal feed composition to the ruminant, as compared to an animal feed composition that does not include the treated fermentation biomass and the treated at least one proteinaceous feed ingredient administered to the ruminant.

According to various embodiments of the methods, the fermentation biomass may be as described herein. For example, according to certain embodiments, the fermentation biomass may be of eukaryotic cell origin, such as a fermentation biomass be selected from the group consisting of a yeast, a yeast cream, a yeast biomass, a lysine biomass, a lactic acid fermentation biomass, a citric acid presscake, an ethanol presscake, distiller's yeast, a brewer's yeast biomass, a baker's yeast biomass, and mixtures or combinations of any thereof. For example, according to certain embodiments, the fermentation biomass may be a yeast selected from the group consisting of brewer's yeast biomass and baker's yeast biomass.

According to other embodiments, the methods may further comprise adding a least one of an organic acid, as described herein, and a gluten protein, as described herein, to the animal feed, and, according to still other embodiments, may further comprise adding one or more ingredient selected from the group consisting of an isolated enzyme, a divalent metal ion, and a plant extract to the animal feed composition. According to these embodiments, the additional ingredients may be added before, during, or after the treating step.

According to certain embodiments of the method, treating the fermentation biomass and the at least one proteinaceous feed ingredient may comprise moist heat treating the fermentation biomass and the at least one proteinaceous feed ingredient. Moist heat treating may comprise heating the fermentation biomass and the at least one proteinaceous feed ingredient with a moisture content from 15% to 50% followed by drying the fermentation biomass and the at least one proteinaceous feed ingredient to a moisture content of 10% to 15%.

According to various embodiments of the methods, treating the fermentation biomass and the at least one proteinaceous feed ingredient may comprise mixing a 50:50 mixture of a fermentation biomass, for example, a yeast, and water; and admixing the mixture with the at least one proteinaceous feed ingredient, wherein the mixture is added in a quantity sufficient to total 15% to 50% moisture of the composition. According to other embodiments, treating the fermentation biomass and the at least one proteinaceous feed ingredient may comprise heating the fermentation biomass and the at least one proteinaceous feed ingredient at a temperature from 87° C. to 116° C. with 15% to 50% moisture for a time of 0.10 hours to 5 hours; and drying the composition to 10% to 15% moisture.

Other embodiments of the methods of feeding an animal may further comprise forming the animal feed composition into a form selected from the group consisting of a treated protein, a treated feed, and a protein supplement. According to certain embodiments where the animal feed composition may be in the form of a protein supplement, wherein the supplement is in a form selected from the group consisting of a meal, a pellet, a block, a tub, a premix, a top-dress, an additive, and a liquid feed supplement.

According to other embodiments, feeding the ruminant the animal feed composition may comprise feeding the ruminant the composition in the form of a supplement in an amount of 0.454 kg/head/day to 3.18 kg/head/day. According to certain embodiments wherein the animal feed composition is in the form of a premix, feeding a ruminant the animal feed composition may comprise feeding the ruminant the premix in an amount of 0.09 kg/head/day to 0.454 kg/head/day.

According to other embodiments, the present disclosure also includes a process for producing a feed supplement. The process, according to these embodiments, comprises: mixing a composition comprising a fermentation biomass of a eukaryotic cell origin and at least one proteinaceous feed ingredient; treating the composition with moist heat; and forming the composition into a form selected from the group consisting of a meal, a pellet, a block, a tub, a premix, an additive, and a liquid feed supplement. According to certain embodiments, the fermentation biomass is of any of the biomasses described herein, for example, a fermentation biomass of eukaryotic origin selected from the group consisting of a yeast, a yeast cream, a yeast biomass, a lysine biomass, a lactic acid fermentation biomass, a citric acid presscake, an ethanol presscake, distiller's yeast, a brewer's yeast biomass, a baker's yeast biomass, and mixtures of any thereof.

According to other embodiments of the process, the process may further comprise mixing an ingredient with the composition. The ingredient may be one or more ingredients selected from the group consisting of an organic acid, a gluten protein, an isolated enzyme, a divalent metal ion, a plant extract and combinations of any thereof, as described herein.

According to various embodiments of the process, mixing the fermentation biomass and the at least one proteinaceous feed ingredient may comprise mixing the fermentation biomass and water, for example, a 10:90 ratio to a 90:10 ratio of the biomass and water; and combining the fermentation biomass and water with the at least one proteinaceous feed ingredient, wherein the mixture is added in a quantity sufficient to total 15% to 50% moisture. According to certain embodiments, the ration may be a 50:50 mixture of the fermentation biomass and water. According to certain embodiments, treating the mixed composition may comprise heating the composition at a temperature from 87° C. to 116° C. with 15% to 50% moisture for a time of 0.10 hours to 5 hours; and drying the composition to 10% to 15% moisture.

According to the various embodiments of the process, the composition may be any for suitable for consumption by the animal, for example, a form selected from the group consisting of a meal, a pellet, a block, a tub, a premix, a top-dress, an additive, and a liquid feed supplement. According to certain embodiments, the composition is in the form of a meal. According to other embodiments, the composition is in the form of a pellet.

The process according to certain embodiments may further comprise placing the composition in a container configured for shipping and associating indicia with the container. The indicia may comprise pictures and/or symbols and words capable of directing a user, for example, on the origin of the composition, brand name of the composition, and/or on how to administer the composition to an animal. Other embodiments of the process may comprise shipping the container, for example, shipping by one or more of truck, airplane, train and/or boat.

According to other embodiments, the present disclosure includes an animal feed composition comprising: a yeast fermentation biomass; and at least one proteinaceous feed ingredient, wherein the yeast fermentation biomass and the at least one proteinaceous feed ingredient have been treated, for example, treated with moist heat, as described herein. According to the embodiments of the animal feed composition, upon administration of the animal feed composition to a ruminant, an amount of protein passing through the rumen of the ruminant (i.e., rumen bypass protein) is increased, as compared to an animal feed composition that does not include the treated yeast fermentation biomass and the treated at least one proteinaceous feed ingredient administered to the ruminant.

According to certain embodiments, the yeast fermentation biomass may be selected from the group consisting of a yeast presscake, a yeast cream, a citric acid biomass, an ethanol biomass, a brewer's yeast biomass, a baker's yeast biomass, and combinations of any thereof. The animal feed composition, according to other embodiments, may further comprise an ingredient selected from the group consisting of an isolated enzyme, a gluten protein, a divalent metal ion, an organic acid, a plant extract, and combinations of any thereof.

The present disclosure also includes various non-limiting method of increasing production in a ruminant. One non-limiting embodiment of such a method comprises feeding to the ruminant a moist heat treated feed composition that comprises one or more of an isolated enzyme, an organic acid, a fermentation biomass, a gluten protein, at least one divalent metal ion, and at least one plant extract according to the various non-limiting embodiments described herein and set forth in the claims.

The various methods and compositions of the non-limiting embodiments of the present disclosure may be fed directly to ruminant animals or added to the ruminant animal's feed as a feed supplement or additive. Ruminant animals which may be fed the compositions of the present disclosure include, but are not limited to, bovines, ovines, and caprines.

The methods according to the various non-limiting embodiments of the present disclosure contemplate feeding the compositions disclosed herein to a ruminant animal, wherein the composition has a physical form as described below. According to these non-limiting embodiments, the physical form of compositions within the present disclosure may be any suitable formulation known in the feed art. For example, suitable formulations include, but are not limited to, treated proteins and feeds, such as, for example, soybean meal, and as a protein supplement in the form of a meal, pellet, block, cube, liquid supplement or feed, agglomeration, premix/additive, mineral, meal, a cooked tub, and a pressed tub formulations. In one non-limiting embodiment, the methods and compositions may comprise a protein supplement with a physical formulation of a meal or pellet formulation that is suitable for direct consumption or as an additive to feed. In another non-limiting embodiment, the physical formulation used in the methods and compositions may comprise a premix that may be admixed into the animal feed prior to consumption by the ruminant.

According to the various non-limiting embodiments of the methods and compositions herein, the amount of the compositions of the present disclosure that may be consumed by the animal varies depending on one or more factors, including, but not limited to, one or more of animal species, age, size, sex, health and production levels. In one non-limiting embodiment, wherein the composition is in the form of a meal or pelletized protein supplement, the method may comprise feeding the compositions of the present disclosure to a ruminant in an amount of from 0.454 kg to 3.18 kg per head per day (kg/head/day) (1.0 to 7.0 lbs/head/day). In another non-limiting embodiment, wherein the composition is utilized as a premix, a non-limiting method may comprise adding the compositions of the present disclosure to animal feed, such that the amount of the compositions consumed by the ruminant is from 0.0454 to 0.454 kg/head/day (0.1 lbs to 1.0 lbs/head/day). In another non-limiting embodiment, the method comprises adding the composition in an amount such that the amount of compositions consumed by the ruminant is from 0.091 kg to 0.136 kg/head/day (0.2 to 0.3 lbs/head/day).

As described herein, the various non-limiting embodiments of the compositions of the present disclosure may be produced by a method comprising: mixing the components of the composition, wherein the composition is as described herein and set forth in the claims; treating the composition at a moisture level of 10% to 50% moisture for 0.10 to 5 hours at a temperature from 87° C. to 116° C.; and drying the composition to a moisture level of 10% to 15% moisture. According to other non-limiting embodiments, the method may further comprise forming the composition into one of a meal, pellet, block, cube, liquid supplement or feed, agglomeration, premix/additive, mineral, meal, a cooked tub, and a pressed tub formulations, using formulation methods known in the art.

In certain non-limiting embodiments wherein the composition comprises one or more isolated enzymes and one or more divalent metal ions, the composition may be produced by a method wherein mixing the components of the composition comprises mixing the composition comprising the one or more isolated enzymes, followed by adding the one or more divalent metal ions and mixing the combined composition. The composition may then be treated with moist-heat, as described above, then formed into any of the forms disclosed herein, such as, a meal, pellet, block, cube, liquid supplement or feed, agglomeration, premix/additive, mineral, meal, a cooked tub, and a pressed tub formulations, using formulation methods known in the art.

In one non-limiting embodiment where the composition is a protein supplement, the composition may be consumed directly by the ruminant animal. In another non-limiting embodiment where the composition is a feed additive or premix, the composition is added to a commercially available feed composition and the feed additive/feed composition mixture is consumed by the ruminant animal.

### EXAMPLES

The following examples illustrate various non-limiting embodiments of the compositions within the present disclosure and are not restrictive of the invention as otherwise described or claimed herein. Unless otherwise noted, all percentage values are weight percentage.

### Example 1

### Rumen Bypass Protein Increase by Addition of an Enzyme

A study was performed to assess the effect of the addition of the enzymes alpha-galactosidase and xylanase on rumen bypass protein content of moist heat treated soybean meal.

This example utilized an artificial rumen fermentation system (Ankom Daisy System, Ankom Technology, Fairport, N.Y.) and a dacron bag technique using 8 treatments as described below. The bags were incubated for time periods of 0, 2, 4, 16, 48, and 72 hours. The milligrams of dry matter and protein (nitrogen×6.25) remaining in the dacron bags was expressed as a percentage of the original weight of dry matter and protein placed in the bags (percent recovery). The results showing RUP content for each treatment is presented in Table 1: Effects of Moist Heat and Enzyme Treatment on Ruminal Degradation of SBM. The percentage of rumen undigested protein (%RUP) for the treatments after each incubation period was calculated by dividing the residual protein amount by the original protein amount and multiplying by 100 to give a percentage value as shown in Table 2: Influence of Rumen Exposure Time on Percent Protein Recovery. The amount of enzyme added to the treatments was 0.005% xylanase or 0.0037% alpha-galactosidase, by weight.

| Trt. No. | Treatment Description |
|---|---|
| 1 | Soybean meal. |
| 2 | Soybean meal heated at 125° C. for 18 hours at 25% moisture. |
| 3 | Soybean meal at 80% moisture heated for 4 hours at 37° C., and |
| | dried to 12% moisture. |
| 4 | Soybean meal with 80% moisture heated for 4 hours at 37° C., |
| | dried to 12% moisture, and then heated at 125° C. for 18 hours |
| | at 25% moisture. |
| 5 | Soybean meal with alpha-galactosidase at 80% moisture heated |
| | for 4 hours at 37° C. and dried to 12% moisture. |
| 6 | Soybean meal with alpha-galactosidase at 80% moisture heated |
| | for 4 hours at 37° C., dried to 12% moisture, and then heated |
| | at 125° C. for 18 hours at 25% moisture. |
| 7 | Soybean meal with xylanase at 80% moisture heated for 4 hours |
| | at 37° C., and dried to 12% moisture. |
| 8 | Soybean meal with xylanase at 80% moisture heated for 4 hours |
| | at 37° C., dried to 12% moisture, and then heated at |
| | 125° C. for 18 hours at 25% moisture. |

TABLE 1
Effects of Moist Heat and Enzyme Treatment on Ruminal Degradation of SBM

| Sample | Trt. 1 | Trt. 2 | Trt. 3 | Trt. 4 | Trt. 5 | Trt. 6 | Trt. 7 | Trt. 8 |
|---|---|---|---|---|---|---|---|---|
| Protein, % of | 55.2 | 55.7 | 58.4 | 58.8 | 56.9 | 57.7 | 61.1 | 56.8 |
| DM | | | | | | | | |
| RUP, | 17.03^{c} | 64.15^{b} | 12.60^{c} | 85.85^{a} | 19.39^{c} | 89.49^{a} | 12.91^{c} | 89.65^{a} |
| % of CP | | | | | | | | |
| Soluble Protein, | 16.22 | 2.12 | 18.77 | 1.66 | 17.00 | 2.55 | 14.48 | 0.95 |
| % of CP | | | | | | | | |

Treatments 4, 6, and 8 (^{a}) are significantly greater than Trt. 2 (^{b}), which is significantly greater than Treatments 1, 3, 5, and 7 (^{c}) at P < .05 by the Student-Newman-Kuels (SNK) a-posteriori test.

The results indicate that the moist heat treatment process, as simulated under laboratory conditions by combining heat (125° C.) and moisture at 25%, had a significant effect on rumen undegraded protein (“RUP”) content of soybean meal (“SBM”) as shown by comparing Treatment 1 (RUP=17%) versus 2 (RUP=64%).

Furthermore, if processing with water (Trt. #3), water and alpha-galactosidase (Trt. #5), and water and xylanase (Trt. #7) was not followed with the moist heat treatment processing conditions, no significant increase in RUP was observed over regular SBM (Trt. #1). In another embodiment of the moist heat treatment methods, if a moist, low-heat (37° C.) pre-treatment was followed by the typical moist heat processing conditions, significant improvements in RUP content were observed, compared with SBM processed without the moist, low-heat pre-treatment.

The enzyme additions of alpha-galactosidase (Trt. #6) and xylanase (Trt. #8) followed by moist heat treatment processing created RUP value of 89.5 and 89.6% of crude protein (“CP”), respectively. These values were numerically better than the 85.9% RUP content observed by pre-treatment with water alone, followed by moist heat treatment processing and significantly better than the RUP values for unprocessed SBM of 17.0% (Trt. #1) and the normal moist heat treatment processing conditions of 64.2% (Trt. #2). In this example, normal moist heat treatment processing (Trt. #2) reduced the protein degradation in the rumen by 69.8%, pre-treating with water before moist heat treatment processing (Trt. #4) 92.5% and adding alpha-galactosidase (Trt. #6)/xylanase (Trt. #8) 94.3%.

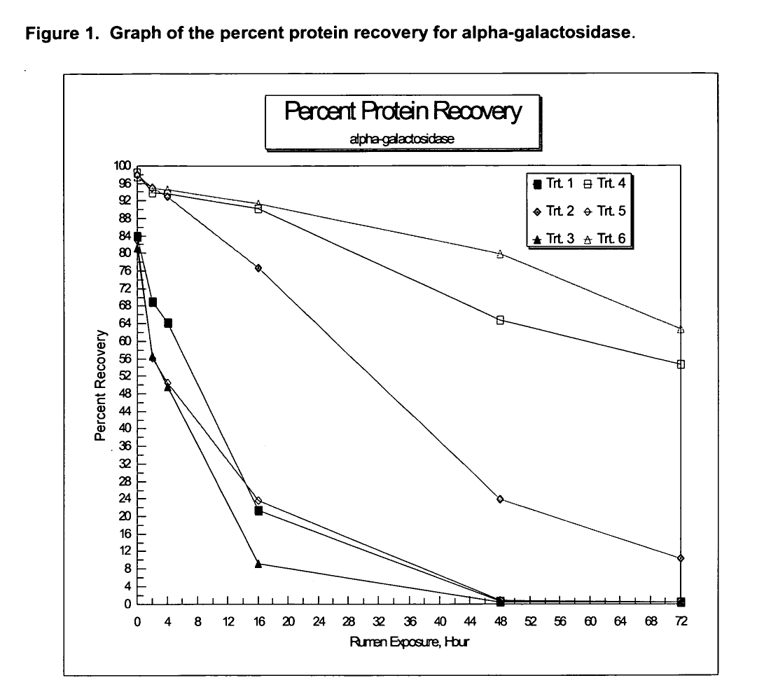

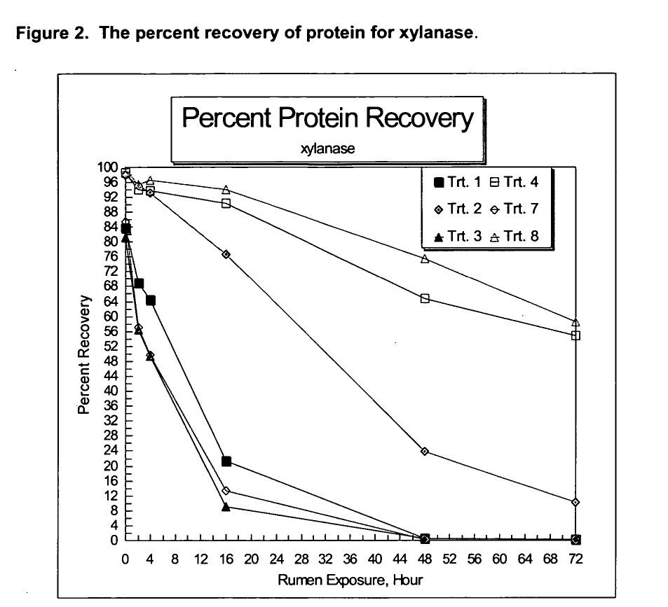

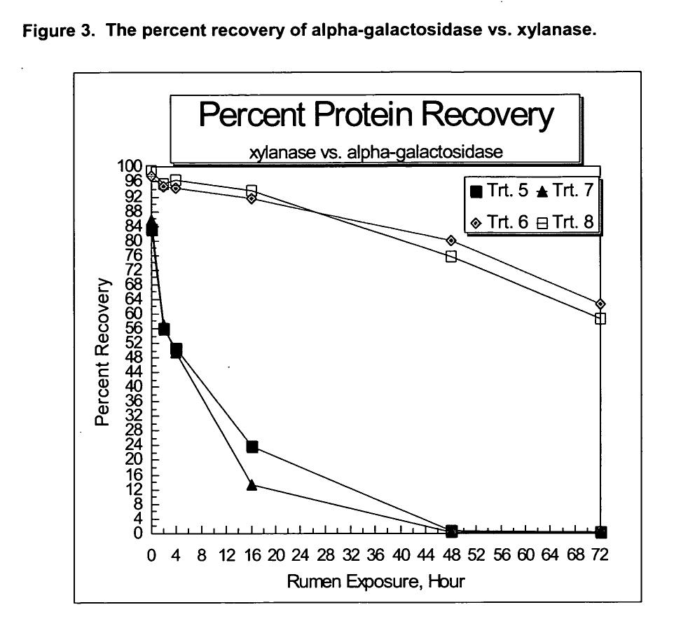

FIGS. 1, 2, and 3 plot the percent protein recovery for compositions comprising alpha-galactosidase and/or xylanase versus rumen exposure time. The figures show that SBM compositions comprising an enzyme and processed with a moist heat treatment method, including the pre-treatment (in Trt. Nos. 6 and 8), show increased amounts of RUP content over the 72 hour testing period.

TABLE 2
Influence of Rumen Exposure Time on Percent Protein Recovery

| | | Incubation | |
|---|---|---|---|
| Trt. No. | Trt. Description | time | % Protein Recovery |
| 1 | Untreated SBM | 0 | 83.78 |
| | | 2 | 69.05 |
| | | 4 | 64.31 |
| | | 16 | 21.27 |
| | | 48 | 0.52 |
| | | 72 | 0.27 |
| 2 | SBM heated | 0 | 97.88 |
| | at 125° C. for 18 hours | 2 | 94.93 |
| | at 25% moisture | 4 | 92.91 |
| | | 16 | 76.61 |
| | | 48 | 23.84 |
| | | 72 | 10.25 |
| 3 | SBM at 80% | 0 | 81.23 |
| | moisture heated for 4 hours at | 2 | 56.52 |
| | 37° C., dried to 12% moisture | 4 | 49.49 |
| | | 16 | 9.05 |
| | | 48 | 0.45 |
| | | 72 | 0.28 |
| 4 | SBM at 80% moisture | 0 | 98.34 |
| | heated for 4 hours at 37° C., | 2 | 93.75 |
| | dried to 12% moisture, and | 4 | 93.51 |
| | then heated at 125° C. for 18 | 16 | 90.20 |
| | hours at 25% moisture | 48 | 64.79 |
| | | 72 | 54.61 |
| 5 | SBM with alpha- | 0 | 83.00 |
| | galactosidase at 80% | 2 | 56.15 |
| | moisture heated for 4 hours | 4 | 50.56 |
| | at 37° C. and dried to 12% | 16 | 23.65 |
| | moisture | 48 | 0.69 |
| | | 72 | 0.34 |
| 6 | SBM with alpha- | 0 | 97.45 |
| | galactosidase at 80% moisture | 2 | 94.70 |
| | heated for 4 hours at 37° C., | 4 | 94.42 |
| | dried to 12% moisture, and | 16 | 91.42 |
| | then heated at 125° C. for 18 | 48 | 79.73 |
| | hours at 25% moisture | 72 | 62.57 |
| 7 | SBM with xylanase at | 0 | 85.52 |
| | 80% moisture heated for 4 | 2 | 56.97 |
| | hours at 37° C. and dried to | 4 | 49.61 |
| | 12% moisture | 16 | 13.38 |
| | | 48 | 0.34 |
| | | 72 | 0.27 |
| 8 | SBM with xylanase at | 0 | 99.05 |
| | 80% moisture heated for 4 | 2 | 95.33 |
| | hours at 37° C., dried to 12% | 4 | 96.57 |
| | moisture, and then heated at | 16 | 93.79 |
| | 125° C. for 18 hours at 25% | 48 | 75.58 |
| | moisture | 72 | 58.55 |

### Example 2

### Effect of Organic Acid

A study was performed to assess the effects of the addition of ascorbic acid and citric acid on bypass protein content of SBM. Samples of SBM were prepared by mixing the SBM with an amount of an organic acid and 25% water (v/w) in a small drum mixer for 3 minutes, treating with moist heat by heating the mixed composition at 105° C. for 4 hours, weighing the samples, and heating at 50° C. for a time sufficient to dry the composition to 12% moisture, as estimated by weight loss.

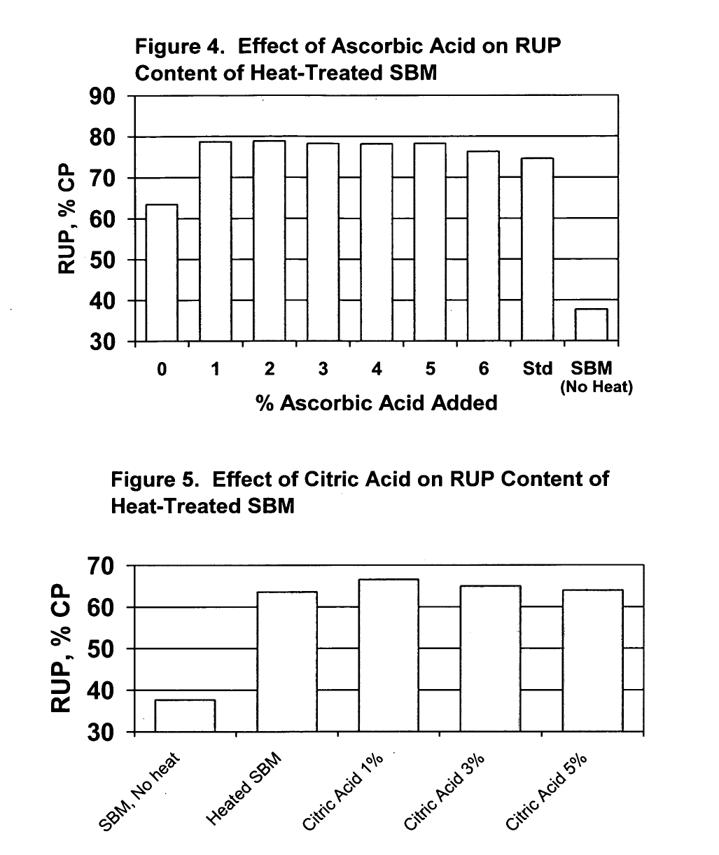

Ascorbic acid was added to the SBM samples in amounts of 0, 1, 2, 3, 4, 5, and 6% (w/w). After processing, RUP was assayed according to the procedure set forth in Example 1. The effect of ascorbic acid on RUP content of the moist heat treated SBM is shown in FIG. 4 and Table 3. Citric acid was added to the high protein SBM in amounts of 1, 3, and 5% (w/w). After processing, RUP was assayed according to the procedure set forth in Example 1. The effect of citric acid on RUP content of the moist heat treated SBM is shown in FIG. 5 and Table 3. Moist heat treated SBM with an organic acid shows increased RUP content when compared to moist heat treated SBM alone.

TABLE 3
Effect of Organic Acid on RUP

| % Added | Fermented | |
|---|---|---|
| Acid | DM, % | RUP, % of CP |
| Ascorbic Acid | | |
| 0 | 59.9 | 63.5 |
| 1 | 52.0 | 78.7 |
| 2 | 52.4 | 78.9 |
| 3 | 52.7 | 78.3 |
| 4 | 53.9 | 78.2 |
| 5 | 52.8 | 78.3 |
| 6 | 54.3 | 76.3 |
| Citric Acid | | |
| 1 | 58.2 | 66.6 |
| 3 | 60.4 | 65.0 |
| 5 | 61.5 | 64.0 |
| Soyhulls | 63.7 | 35.8 |
| Soybean Meal | 75.9 | 37.7 |
| Std Error | 1.1 | 1.7 |

### Example 3

### Effect of Enzyme, Yeast and Metal

A study was performed to examine the effect on the RUP content of the moist heat treated combination of an ethanol yeast biomass, an enzyme, and metal ions. An ethanol yeast biomass was examined alone or in combination with 6000 ppm of a blend of divalent zinc and manganese ions, or 0.01% of xylanase enzyme. The combined mixture was treated with moist heat according to the method set forth in Example 2. The RUP content of the resulting supplement was measured by the standard method set forth in Example 1 (16 hours in situ fermentation) and compared against SBM that had been moist heat treated and combined with an enzyme and metal ions.

The results of the study, shown in Table 4: RUP (% of CP) of Heat-Treated SBM and Ethanol Yeast Biomass, demonstrate that the moist heat treated combination of an ethanol yeast biomass with xylanase displays higher RUP than the moist heat treated ethanol yeast biomass itself. The moist heat treated combination of SBM with a biomass and an enzyme or at least one metal ion may show further improved RUP content, compared to the moist heat treated SBM with an enzyme or at least one metal.

TABLE 4
RUP (% of CP) of Heat-Treated SBM
and Ethanol Yeast Biomass

| | SBM | Yeast |
|---|---|---|
| Unheated | 37.2 | 0.3 |
| Heated | 68.4 | 10.7 |
| +Enzyme | 64.8 | 12.4 |
| +Metals | 66.3 | 5.1 |

0.01% added commercial xylanase, 3000 ppm each of Zn and Mn

### Example 4

### Comparison of Xylanases

A study was performed to examine the effect of two varieties of xylanase enzyme on the production of bypass protein in SBM. The varieties of xylanase studied were xylanase Thermomyces lanuginosus (temperature resistant xylanase) and xylanase cocktail (a combination of xylanase, hemicellulase, cellulose, and alpha-galactosidase available from D.F. International, LLC, Gaithersburg, Md.). The xylanase was added to SBM in amounts of 0.05, 0.1, 0.2, 0.4 grams enzyme per kilogram (0.1, 0.2, 0.4, and 0.8 pounds enzyme per ton) of SBM and the samples processed with the moist-heat treatment method as described in Example 2.

The results are presented in Table 5: Effect of Xylanase on RUP Content of SBM. The results show that xylanase T. lanuginosus provided minimal increase in RUP content when added in amounts under 0.8 lb/tonSBM. However, the xylanase cocktail displayed improved RUP content compared to the control. Significant improvement in RUP content was observed at xylanase contents of 0.05 to 0.4 g/kg (0.1 to 0.4 lb/ton)SBM.

TABLE 5
Effect of Xylanase on RUP Content of SBM

| | Concentrations of Enzymes | | | | |
|---|---|---|---|---|---|
| | 0.05 g/kg | 0.1 g/kg | 0.2 g/kg | 0.4 g/kg | Control |
| RUP, % of CP | 71.45 | 71.91 | 70.57 | 73.16 | 72.09 |
| Xylanase | | | | | |
| Thermomyces | | | | | |
| lanuginosus Enzyme | | | | | |
| RUP, % of CP | 74.11 | 79.04 | 73.57 | 70.4 | 71.37 |
| Xylanase Cocktail | | | | | |
| Enzyme | | | | | |

### Example 5

### Effect of Xylanase and Metals

A study was performed to assess whether the combination of SBM with an enzyme and metal ions would show further improved RUP contents, compared to a metal ion/SBM mixture. The compositions were moist heat treated according to the method disclosed in Example 2, with the following modification. To allow more time for the enzymes to interact with the SBM, a pre-batch was made as follows: a dosage of 0.1 g enzyme/kg SBM (0.2 lb/ton) of D.F. International xylanase cocktail was added along with 80% of the added water to the SBM and the composition mixed for 30 minutes at 80° C. prior to the addition of the three metals and the remaining water. The metal treatments were evaluated at 0, 125, 250, 500,1000, and 2000 ppm each of Zn, Mn, and Fe (ferrous state). The metals were made soluble in the SBM/Xylanase mixture and mixed for 3 minutes. The samples were then cooked for 5 hours at 105° C. After the samples were prepared, the RUP values were determined in situ using according to the procedure in Example 1. The results are shown in the Table 6: Enzyme/Metal Composition in Effect on RUP Content.

TABLE 6
Enzyme/Metal Composition Effect on RUP Content

| Tri Metal, | With Enzyme | W/O Enzyme |
|---|---|---|
| (ppm of total metal) | RUP, % of CP | RUP, % of CP |
| 0 | 54.4 | 54.4 |
| 125 | 60.6 | |
| 250 | 57.4 | 61.1 |
| 500 | 61.7 | 66.0 |
| 1000 | 68.1 | 63.5 |
| 2000 | 69.0 | 64.9 |

The xylanase cocktail, used in a treatment concentration of 0.1 g enzyme/kg SBM (0.2 lb/ton), in combination with ferrous iron, zinc, and manganese ions has a positive effect on the RUP percent content of SBM when the metal concentrations are 1000 ppm to 2000 ppm each (3000 ppm to 6000 ppm total metal ion concentration).

### Example 6

### Combination of Organic Acid and Metals

A series of three studies were performed to assess the use of organic acids, alone and in combination with metal ions, on the RUP content of moist heat treated SBM. The SBM mixtures were prepared and moist heat treated according to the method disclosed in Example 2.

In a first study, ascorbic acid was added to the SBM in amounts of 0, 0.1, 0.25, 0.5, 0.75, 1.0, 1.5, and 2.0% (w/w) to determine minimum ascorbic acid levels needed to increase RUP content of moist heat treated composition compared to the moist heat treated SBM control. In addition to the standard procedure, treatments were evaluated after 4 and 5 hours of heating to evaluate the interaction of heating time and sensitivity to treatment differences.

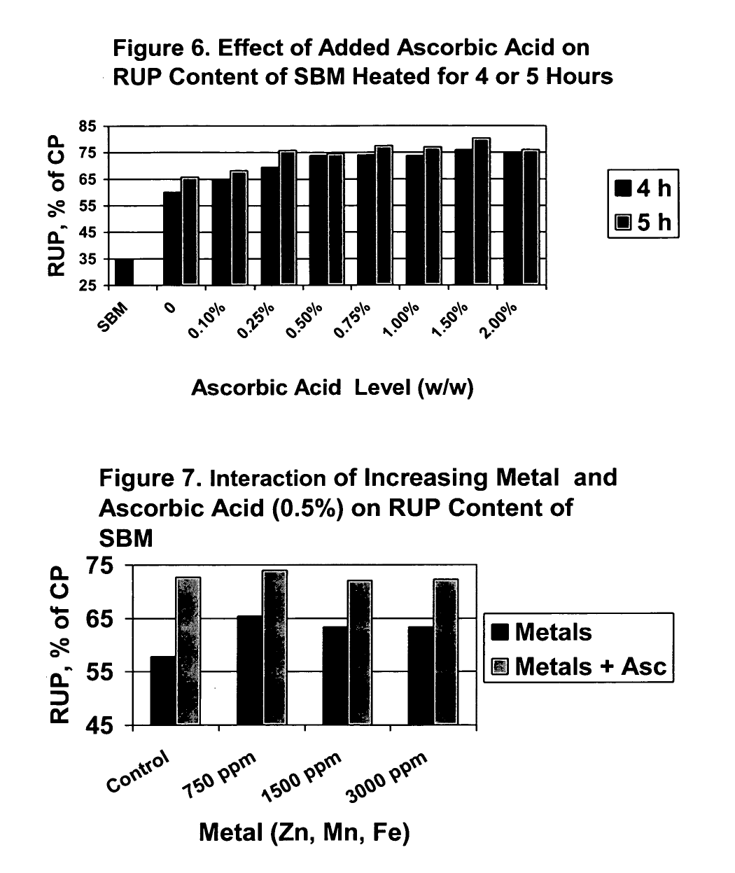

The inclusion of ascorbic acid increased RUP beyond the levels for RUP of the moist heat treated SBM control for all the levels investigated in this test, as shown in Table 7: Effect of Ascorbic Acid and Heating Time on RUP of SBM and FIG. 6: Effect of Added Ascorbic Acid on RUP Content of SBM Heated for 4 or 5 hours. After 4 hours of heating at 105° C., the inclusion of 0.1% ascorbic acid elevated RUP about 5% above the control. RUP content was increased by about 14% with ascorbic acid inclusions from 0.5 to 2%. Increased heating time to 5 hours at 105° C. further elevated the average RUP content of the samples.

In a second study, the interactions of ascorbic acid and metal ions were assessed. In a first test, zinc, manganese, and ferrous iron metal ions were added to the SBM at 750, 1500, and 3000 ppm total metal concentration, i.e., each metal was added in concentrations of 250, 500 and 1000 ppm respectively, with the addition of 0.5% (w/w) ascorbic acid in half of the samples. The results are presented in Table 8: Effect of Metal Addition on the Enhancement of RUP Elicited by Ascorbic Acid and FIG. 7: Interaction of Increasing Metal and Ascorbic Acid (0.5%) on RUP Content of SBM. Both the metal ions and the metal ions plus ascorbic acid showed increased RUP content relative to the moist heat treated SBM control, with the metal ion/ascorbic acid combination having the higher RUP content.

TABLE 7
Effect of Ascorbic Acid and Heating Time on RUP of SBM

| 4 hours heating at 105° C. | | | | | | | | | | |
|---|---|---|---|---|---|---|---|---|---|---|
| Ascorbic Acid Added, % by weight | | | | | | | | | | |
| Item¹ | SBM | 0 | 0.1 | 0.25 | 0.5 | 0.75 | 1 | 1.5 | 2 | SE |
| Moisture, % | 10.3 | 13.3 | 13.1 | 13.4 | 13.5 | 13.8 | 13.9 | 13.8 | 14.0 | 0.3 |
| CP, % | 48.5 | 47.6^{abc} | 48.2^{bc} | 48.6^{c} | 47.6^{abc} | 47.4^{abc} | 47.0^{a} | 47.5^{abc} | 46.4^{a} | 0.4 |
| SIP, % of CP | 9.86 | 7.56 | 7.68 | 8.25 | 6.03 | 7.60 | 6.74 | 7.36 | 5.35 | 1.1 |
| RUP, % of CP | 34.8 | 60.0^{a} | 64.6^{b} | 69.5^{c} | 73.9^{cd} | 74.0^{cd} | 73.7^{cd} | 75.9^{d} | 74.9^{d} | 1.4 |

| 5 hours heating at 105° C. | | | | | | | | | |
|---|---|---|---|---|---|---|---|---|---|
| Ascorbic Acid Added, % by weight | | | | | | | | | |
| Item | 0 | 0.1 | 0.25 | 0.5 | 0.75 | 1 | 1.5 | 2 | SE |
| Moisture, % | 12.6 | 11.6 | 11.8 | 12.4 | 12.6 | 12.9 | 11.7 | 12.4 | 0.5 |
| CP, % | 48.2 | 48.8 | 48.2 | 47.9 | 48.3 | 47.8 | 48.0 | 47.7 | 0.4 |
| SIP, % of CP | 9.00^{b} | 82.6^{ab} | 6.16^{a} | 6.06^{a} | 8.48^{ab} | 7.10^{ab} | 7.12^{ab} | 8.38^{ab} | 0.8 |
| RUP, % of CP | 65.8^{a} | 68.2^{a} | 75.8^{b} | 74.6^{b} | 77.5^{bc} | 77.0^{bc} | 80.3^{c} | 76.0^{b} | 1.1 |

¹SIP = Soluble intake protein, RUP = rumen undegraded protein
^{a,b,c,d}Means within row with different superscripts differ (P < .05)

TABLE 8
Effect of Metal Addition on the Enhancement of RUP Elicited by Ascorbic Acid

| | Metals Added, ppm of Total Metal | | | | | | | | | |
|---|---|---|---|---|---|---|---|---|---|---|
| | 0 | 750 | 1500 | 3000 | 0 | 750 | 1500 | 3000 | | |
| Item¹ | | No Ascorbic acid | | | 0.5% Ascorbic Acid | | | | SE | SBM |
| Moisture, % | 12.8 | 13.6 | 14.0 | 14.3 | 14.2 | 15.0 | 14.0 | 14.6 | 0.8 | 9.0 |
| CP, % of DM | 55.1 | 54.4 | 55.6 | 54.8 | 54.8 | 54.4 | 55.3 | 55.0 | 0.4 | 54.1 |
| SIP, % of CP | 6.78^{a} | 6.84^{a} | 7.61^{a} | 6.01^{ab} | 3.72^{bc} | 2.84^{c} | 5.70^{a} | 6.28^{ab} | 0.9 | 8.5 |
| RUP, % of CP | 57.8^{a} | 65.4^{b} | 63.4^{b} | 63.4^{b} | 72.7^{c} | 74.0^{c} | 72.1^{c} | 72.3^{c} | 1.5 | 30.7 |

¹DM = Dry matter, SIP = Soluble intake protein, RUP = rumen undegraded protein
^{a,b,c}Means within row with different superscripts differ (P < .05)

TABLE 9
Effect of Ascorbic Acid on the Enhancement of RUP Elicited by Metal

| | Ascorbic Acid Added, wt:wt % | | | | | | | | | |
|---|---|---|---|---|---|---|---|---|---|---|
| | 0 | 0.25 | 0.5 | 1.0 | 0 | 0.25 | 0.5 | 1.0 | | |
| Item¹ | No Metal Addition | | | | 1500 ppm Metals added | | | | SE | SBM |
| Moisture, % | 12.3^{a} | 13.2^{ab} | 13.9^{ab} | 13.6^{bc} | 13.2^{ab} | 14.6^{c} | 13.4^{ab} | 13.9^{bc} | 0.3 | 9.0 |
| CP, % of DM | 54.4 | 54.7 | 53.4 | 53.6 | 54.6 | 54.6 | 55.2 | 54.7 | 0.6 | 54.1 |
| SIP, % of CP | 7.35 | 5.44 | 3.60 | 4.61 | 4.87 | 4.97 | 6.08 | 6.00 | 1.8 | 10.2 |
| RUP, % of CP | 61.9^{a} | 68.0^{bc} | 72.6^{cd} | 73.0^{d} | 67.3^{b} | 72.3^{cd} | 74.9^{d} | 73.4^{d} | 1.4 | 28.0 |

¹DM = Dry matter, SIP = Soluble intake protein, RUP = rumen undegraded protein
^{a,b,c,d}Means within row with different superscripts differ (P < .05)

TABLE 10
Interaction of Citric Acid and Metal on the RUP Content of SBM

| | Metal Added, 1000 ppm | | | | | | | | |
|---|---|---|---|---|---|---|---|---|---|
| | None | Zn | Fe⁺² | Fe⁺³ | None | Zn | Fe⁺² | Fe⁺³ | |
| Item¹ | No Citric Acid | | | | 1% Citric Acid Added | | | | SE |
| Moisture, % | 14.2^{bc} | 14.0^{abc} | 13.9^{abc} | 15.6^{d} | 14.4^{c} | 13.0^{a} | 13.2^{ab} | 14.0^{abc} | 0.3 |
| CP, % of DM | 54.7 | 54.6 | 54.8 | 54.8 | 54.4 | 53.0 | 53.7 | 54.8 | 0.6 |
| SIP, % of CP | 4.86 | 7.44 | 4.24 | 4.52 | 4.36 | 2.00 | 2.52 | 3.88 | 1.7 |
| RUP, % of CP | 61.0^{ab} | 56.1^{a} | 67.8^{bc} | 65.6^{abc} | 60.3^{ab} | 56.6^{a} | 72.4^{c} | 59.0^{ab} | 3.4 |

¹DM = Dry matter, SIP = Soluble intake protein, RUP = rumen undegraded protein
^{a,b,c,d}Means within row with different superscripts differ (P < .05)

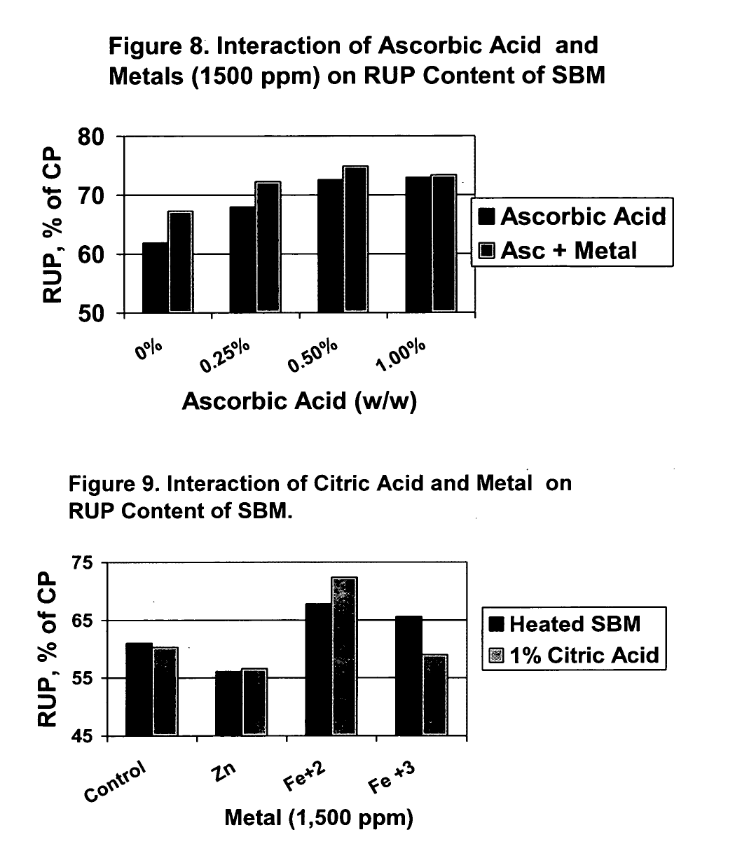

In a second test, ascorbic acid was added to the SBM at 0.25, 0.5, and 1.0% (w/w) with the addition of 1500 ppm of the metal ion combination to half the samples. The results are presented in Table 9: Effect of Ascorbic Acid on the Enhancement of RUP Elicited by Metal and FIG. 8: Interaction of Ascorbic Acid and Metals (1500 ppm) on RUP Content of SBM. Both the moist heat treated SBM with ascorbic acid and the moist heat treated SBM with ascorbic acid plus metal ions showed increased RUP content relative to the moist heat treated SBM control. Greater increases were seen for the moist heat treated SBM with both ascorbic acid and metal ions. Samples with ascorbic acid alone showed no significant increase above the addition of 0.5% of the acid. The ascorbic acid plus metal ions showed no significant differences among each of the treatments, such that 0.25% ascorbic acid in combination with metals was as effective as 0.5% ascorbic acid alone.

In a third study, the effect of citric acid (1% w/w), alone or in combination with 1000 ppm of zinc ions, ferrous iron ions, or ferric iron ions, on RUP content of moist heat treated SBM was examined and compared to the effects of the metal species alone. The results of the study are presented in Table 10: Interaction of Metal and Citric Acid on the RUP Content of SBM and FIG. 9: Interaction of Metal and Citric Acid on the RUP Content of SBM. Both citric acid and the metal treatments alone did not significantly increase the RUP content of the SBM above the levels observed in the control. However, the combination of citric acid and ferrous iron ions in moist heat treated SBM showed significant improvement in RUP content compared to the control or SBM with either citric acid or ferrous iron ions.

### Example 7

### Combined Effect of Xylanase and Plant Extract

In this study the effect of yucca and quillaja saponins on RUP content of SBM was assessed. The saponins effect on RUP content was assessed both individually and in combination with xylanase enzyme.

SBM samples were mixed in a small drum-type mixer for 3 minutes with the treatment and 25% water (w/w). Yucca and quillaja saponins were incorporated into the added water in amounts of 0.5% and 1% w/w. The plant extracts were incorporated into the SBM either alone or in combination with xylanase enzyme (D.F. International, Gaithersburg, Md.) in amounts of 0.05 g enzyme/kg SBM (0.1 lb/ton) and 0.1 g enzyme/kg SBM (0.2 lbs/ton). The SBM sample mixtures were then process by moist heat treatment according to the method set forth in Example 2. The RUP content for each sample was measured using the protocol set forth in Example 1 and compared to un-heated and moist heat treated SBM control samples. The results are set forth in Table 11: Effect of Xylanase and Saponin on RUP content of SBM. Enzyme addition at the 0.1 lb/ton rate reduced RUP content, but was beneficial at the 0.1 g/kg rate and 1% quillaja saponin.

### Example 8

### Effect of Corn Gluten Meal and Metal

In this study the effect of corn gluten meal on RUP content of SBM was assessed. The effect of corn gluten meal on RUP content was assessed both individually and in combination with the zinc, manganese and ferrous iron metal ion combination.

The SBM samples were mixed in a small drum-type mixer for 3 minutes with the treatment. Corn gluten meal treatments were added to the sample mixtures in amounts of 1%, 2.5%, 5%, 10%, 15%, and 20% (w/w). The gluten meal treatments were evaluated individually and in combination with 1500 ppm of a metal ion mixture (500 ppm each of zinc, manganese and ferrous iron metal ion). The SBM sample mixtures were then processed by moist heat treatment according to the method set forth in Example 2. The RUP content for each sample was measured using the protocol set forth in Example 1 and compared to un-heated and moist heat treated SBM control samples. The results of the study are presented in Table 12: Effect of Corn Gluten Meal and Metals on RUP of SBM.

The addition of corn gluten meal showed significant effect on RUP content at addition levels of 15% to 20% when compared to the moist heat treated SBM control. The addition of metal to the corn gluten meal/SBM mixture showed increased RUP content for corn gluten meal when compared to the corn gluten meal/SBM mixture alone.

TABLE 11
Effect of Xylanase¹ and Saponin on RUP of Soybean Meal

| | | No Xylanase | | | | | Xylanase, 0.05 g/kg | | | | |
|---|---|---|---|---|---|---|---|---|---|---|---|
| | Unheated | None | Yucca | | Quillaja | | None | Yucca | | Quillaja | |
| Item² | SBM | 0 | 0.5% | 1% | 0.5% | 1% | 0 | 0.5% | 1% | 0.5% | 1% |
| Moisture, % | 11.0^{a} | 11.2^{ab} | 12.3^{abcde} | 13.2^{cdef} | 13.4^{def} | 12.8^{bcdef} | 13.4^{def} | 13.6^{ef} | 12.8^{bcdef} | 12.8^{bcdef} | 14.0^{f} |
| CP % | 49.5^{e} | 48.8^{de} | 47.9^{abcd} | 47.2^{ab} | 47.6^{ab} | 47.6^{abc} | 47.8^{abcd} | 47.4^{ab} | 47.6^{abc} | 47.9^{abcd} | 46.9^{a} |
| SIP, % of CP | 15.4^{d} | 8.7^{bc} | 9.4^{c} | 6.1^{a} | 8.4^{abc} | 7.2^{abc} | 8.9^{bc} | 8.8^{bc} | 7.1^{abc} | 8.2^{abc} | 6.7^{ab} |
| RUP, % of CP | 27.3^{a} | 66.9^{bc} | 60.3^{bc} | 63.2^{bc} | 65.2^{bc} | 60.2^{bc} | 60.5^{bc} | 60.4^{bc} | 58.9^{b} | 61.6^{bc} | 62.3^{bc} |

| | | Xylanase, 0.1 g/kg | | | | | |
|---|---|---|---|---|---|---|---|
| | Unheated | None | Yucca | | Quillaja | | |
| Item² | SBM | 0 | 0.5% | 1% | 0.5% | 1% | SE |
| Moisture, % | 11.0^{a} | 11.6^{abc} | 12.0^{abcde} | 11.9^{abcd} | 13.2^{cdef} | 12.1^{abcde} | 0.7 |
| CP % | 49.5^{e} | 48.6^{cde} | 48.0^{bcd} | 48.1^{bcd} | 47.8^{abcd} | 48.0^{bcd} | 0.4 |
| SIP, % of CP | 15.4^{d} | 7.6^{abc} | 8.2^{abc} | 6.3^{a} | 8.4^{abc} | 7.3^{abc} | 1.0 |
| RUP, % of CP | 27.3^{a} | 66.3^{bc} | 65.5^{bc} | 62.2^{bc} | 61.7^{bc} | 67.7^{c} | 3.3 |

¹Xylanase supplied by DF International
²SIP = Soluble intake protein, RUP = rumen undegraded protein
^{a,b,c,d,e,f}Means within row with different superscripts differ (P < .10)

TABLE 12
Effect of Corn Gluten Meal and Metal on RUP of SBM

| | | No Metals Added | | | | | | | |
|---|---|---|---|---|---|---|---|---|---|
| | Unheated | | Corn Gluten Meal, Addition, % | | | | | | |
| Item¹ | SBM | SBM | 1% | 2.5% | 5% | 10% | 15% | 20% | CGM |
| Moisture, % | 11.6^{a} | 13.6^{a} | 13.4^{a} | 14.2^{ab} | 14.6^{ab} | 14.2^{ab} | 12.9^{a} | 14.8^{ab} | 14.4^{ab} |
| CP % | 49.5^{bcde} | 48.1^{ab} | 48.6^{bcd} | 48.2^{ab} | 48.4^{abc} | 49.4^{bcde} | 50.0^{de} | 50.2^{e} | 58.2^{f} |
| SIP, % of CP | 18.8^{ab} | 9.7^{a} | 8.8^{a} | 8.5^{a} | 9.5^{a} | 10.6^{a} | 8.5^{a} | 9.8^{a} | 10.1^{a} |
| RUP, % of CP | 21.3^{a} | 63.0^{bcd} | 60.0^{b} | 60.0^{b} | 62.0^{bc} | 64.4^{bcd} | 71.4^{ef} | 68.7^{de} | 81.0^{g} |

| | | 1500 ppm Metals Added, (500 each Zn, Mn, Fe) | | | | | | | |
|---|---|---|---|---|---|---|---|---|---|
| | Unheated | | Corn Gluten Meal, Addition, % | | | | | | |
| Item¹ | SBM | SBM | 1% | 2.5% | 5% | 10% | 15% | 20% | CGM |
| Moisture, % | 11.6^{a} | 11.8^{a} | 16.8^{b} | 13.4^{a} | 14.2^{ab} | 14.2^{ab} | 14.6^{ab} | 13.6^{a} | 13.3^{a} |
| CP % | 49.5^{bcde} | 49.1^{bcde} | 47.2^{a} | 48.3^{abc} | 48.4^{abc} | 48.8^{bcd} | 49.6^{cde} | 50.4^{e} | 59.0^{f} |
| SIP, % of CP | 18.8^{ab} | 8.2^{a} | 10.1^{a} | 8.1^{a} | 7.7^{a} | 8.1^{a} | 7.6^{a} | 38.7^{b} | 10.0^{a} |
| RUP, % of CP | 21.3^{a} | 69.2^{de} | 67.3^{cde} | 64.1^{bcd} | 61.3^{bc} | 68.0^{cde} | 67.9^{cde} | 77.3^{fg} | 78.4^{g} |

¹SIP = Soluble intake protein, RUP = rumen undegraded protein
^{a,b,c,d,e,f,g}Means in the same column with different superscripts are different (P < .10).

### Example 9

### Use of Fermentation Biomass

In this example, the effect of a liquid brewer's yeast (10.6% dry matter) on the formation of RUP content in a simulated moist-heat manufacturing process using SBM as the proteinaceous substrate was evaluated.

SBM was mixed for three minutes in a small mixer with brewer's yeast and water. Added moisture varied from 10% to 35% (v/w) with brewer's yeast providing 25% to 100% of the total added moisture within moisture level. The treatment design resulted in brewer's yeast dry matter comprising 0.33% to 4.60% of the dry weight of treated material. Treated material was heated in a closed container at 105° C. for four hours after which samples were oven-dried at 50° C. to a final moisture content of 12%. The RUP content of samples was determined as set forth in Example 1.

The results of this study are presented in Table 13: Effects of Brewer's Yeast on Rumen Undegradable Protein Content. For the conditions of this example, adding 0.50% to 2.30% brewer's yeast on a dry matter basis under conditions of 15% to 25% added moisture resulted in 79.4% to 83.2% RUP content of treated material. At 35% added moisture, brewer's yeast appeared to be less effective at causing RUP formation, although values between 74.4% and 79.8% RUP were observed. For treatments containing only 10% added moisture, there appeared to be benefits for adding higher concentrations of brewer's yeast. The highest RUP content (83.3%) occurred when brewer's yeast comprised 0.99% of the mixture and at 15% total added moisture.

TABLE 13
Effects of Brewer's Yeast on the RUP Content of SBM Moist-Heat Treated
Under Varying Amounts of Added Moisture

| Brewers Yeast | | | | | | Brewers Yeast |
|---|---|---|---|---|---|---|
| % of total added | Added Moisture | | | | | Grams of DM/315 g |
| moisture | 10% | 15% | 20% | 25% | 35% | DM SBM |
| 25 | 68.42^{B} | | | | | 1.04 (0.33%) |
| 25 | | 80.900^{A} | | | | 1.56 (0.50%) |
| 25 | | | 81.525^{A} | | | 2.07 (0.66%) |
| 50 | 80.600^{A} | | | | | 2.07 (0.66%) |
| 25 | | | | 80.825^{A} | | 2.59 (0.82%) |
| 50 | | 83.275^{A} | | | | 3.11 (0.99%) |
| 25 | | | | | 76.100^{AB} | 3.63 (1.15%) |
| 100 | 81.750^{A} | | | | | 4.15 (1.32%) |
| 50 | | | 79.425^{A} | | | 4.15 (1.32%) |
| 50 | | | | 80.800^{A} | | 5.19 (1.65%) |
| 100 | | 82.675^{A} | | | | 6.22 (1.97%) |
| 50 | | | | | 79.775^{A} | 7.25 (2.30%) |
| 100 | | | 82.150^{A} | | | 8.30 (2.63%) |
| 100 | | | | 80.850^{A} | | 10.37 (3.29%) |
| 100 | | | | | 74.400^{AB} | 14.50 (4.60%) |

Brewer's Yeast comprising 10.6% dry matter (“DM”) was applied to the SBM at 100%, 50% and 25% of the total amount of added moisture. The remaining percentages of added moisture were comprised of water. The grams of yeast applied to the grams of SBM on a dry matter basis and the percentage yeast in the total mass on a dry matter basis are listed in the right column of the table.
^{A,AB,B}Means with the same letter are not statistically different (P < 0.05).

### Example 10

### Fermentation Biomass in Combination with Other Components

In this example, a series of studies was undertaken to evaluate the effectiveness of fermentation biomasses, alone or in combination with other components, to assist in the formation of RUP content in a moist-heat manufacturing process. The components were added to SBM and the percentage RUP content was measured using the procedure as described in Example 1. In each study, the biomass was added to the SBM in amounts that provides 25% and/or 35% total moisture in the composition.

An initial screen evaluated the effectiveness of various fermentation biomasses in increasing the formation of RUP content if moist-heat treated SBM. Comparison controls of SBM, moist-heat processed SBM, and soy hulls were used. The results of the study are presented in Table 14: In Vitro Screen of Moist-Heat Treated SBM as Affected by Biomass Addition.

TABLE 14
In Vitro Screening of Moist-Heat Treated SBM as
Affected by Biomass or Soy hull Addition

| Treatment | DMD¹, % | RUP, % of CP |
|---|---|---|
| Soybean Meal | 78.5^{e} | 34.5^{a} |
| Heated Soybean Meal | 60.6^{cd} | 64.6^{bcde} |
| SBM plus Brewer's Yeast | 42.4^{a} | 86.3^{g} |
| SBM plus Citric Acid Yeast Cream | 59.9^{cd} | 65.9^{bcde} |
| SBM plus Corn Steep Liquor | 61.0^{cd} | 69.1^{cde} |
| SBM plus Ethanol Distillers Solubles | 58.8^{c} | 69.4^{cdef} |
| SBM plus Lactic Acid Biomass | 54.2^{b} | 74.1^{ef} |
| SBM plus Lysine Biomass | 59.7^{cd} | 63.4^{bc} |
| SBM plus 5.5% Soy Hulls | 61.1^{cd} | 64.6^{bcde} |
| SBM plus 11% Soy Hulls | 61.6^{d} | 62.7^{bc} |
| SE | 0.9 | 1.6 |

¹DMD = dry matter disappearance
^{a,b,c,d,e}Means within column with different superscript are different (P < .05)

In a second study, wet fermentation biomasses were added to SBM to obtain 25% and 35% added moisture in the moist-heat treatment process. The results of this study are presented in Table 15: Effect of Addition Rate of Biomass on RUP Content of Moist-Heat Treated SBM. In the treatments, increased moisture tended to decrease the formation of RUP. The addition of lactic acid biomass or the brewer's yeast biomass increased RUP content relative to the moist-heat processed SBM control.

TABLE 15
Effect of Addition Rate¹ of Biomass on RUP Content
in Moist-Heat Treated SBM

| Addition Level | 25% | 35% | Average |
|---|---|---|---|
| TREATMENT | | | |
| Heated Soybean Meal | 64.6^{bc} | 65.6^{C} | 65.1^{c} |
| SBM plus Brewer's Yeast | 81.1^{e} | 76.5^{d} | 78.8^{e} |
| SBM plus Citric Acid Yeast Cream | 63.0^{bc} | 67.4^{c} | 65.2^{c} |
| SBM plus Corn Steep Liquor | 57.6^{a} | 42.7^{a} | 50.2^{a} |
| SBM plus Ethanol Distillers Solubles | 64.3^{bc} | 56.3^{b} | 60.3^{b} |
| SBM plus Lactic Acid Biomass | 69.6^{d} | 68.1^{c} | 68.9^{d} |
| SBM plus Lysine Biomass | 60.2^{ab} | 54.0^{b} | 57.1^{b} |
| SBM plus 5.5% Soy Hulls | 67.3^{cd} | 66.9^{c} | 67.1^{cd} |
| SBM plus 11% Soy Hulls | 66.3^{cd} | 64.1^{c} | 65.2^{c} |
| SE | 1.7 | 1.7 | 1.2 |

¹Biomass added at a rate sufficient to contribute 25 or 35% added moisture.
^{a,b,c,d,e}Means within column with different superscripts are different (P < .05).

In a third study, the affect of the combination of the various fermentation biomasses and enzymes was assessed. In this study, the biomasses were combined with a combination of xylanase and heat-stable amylase (0.1 g/kg of xylanase and 100 k units of heat-stable alpha-amylase). The results are presented in Table 16: Effect of Biomass and Enzymes Addition on RUP Content of Moist-Heat Treated SBM. On average, the combination of biomass and enzymes showed increased RUP content.

TABLE 16
Effect of Biomass and Enzymes* Addition on RUP
(% Of CP) of Moist-Heat Treated SBM

| Enzymes | No | Yes | Average |
|---|---|---|---|
| TREATMENT | | | |
| Heated Soybean Meal | 64.2 | 64.3 | 64.2^{b} |
| SBM plus Brewer's Yeast | 81.2 | 80.3 | 80.8^{c} |
| SBM plus Lactic Acid Biomass | 65.9 | 66.3 | 66.1^{b} |
| SBM plus Ethanol Distillers Solubles | 61.4^{x} | 67.1^{y} | 64.2^{b} |
| SBM plus Citric Acid Yeast Cream | 62.8 | 62.6 | 62.7^{b} |
| SBM plus Lysine Biomass | 54.8 | 56.3 | 55.6^{a} |
| SBM plus 5.5% Soy Hulls | 62.1 | 65.1 | 63.6^{b} |
| Mean | 64.6 | 66.0 | |

^{a,b,c}Means within columns with different superscripts differ (P < .05).
^{x,y}Means within row with different superscripts differ (P < .10).
*Biomass treated with 0.1 g/kg xylanase and 100 k units heat stable α amylase for 30 minutes before addition to SBM. For treated SBM without added biomass, enzyme was added directly to SBM.

In a fourth study, the affect of the combination of the various fermentation biomasses with a combination of metals on RUP content of SBM was assessed. The metals zinc, manganese, and ferrous iron were used in amounts of 500 ppm each (1500 ppm total metal addition), while the biomass was added in quantities to obtain 25% and 35% added moisture in the moist-heat treatment process. The results of the study are presented in Table 17: Interaction of Metals and Biomass Inclusion Rate on RUP Content of Moist-Heat Treated SBM. The combination of metals and biomass showed increased RUP content at both biomass addition levels.

In addition, there was a significant interaction between citric acid and the added metals. Citric acid fermentation biomass alone provided little benefit relative to moist-heat treated SBM, however, the combination of citric acid biomass and the metals showed more RUP content than either the citric acid biomass or the metals alone.

TABLE 17
Interaction of Metals* and Biomass Inclusion Rate on RUP of Moist-Heat Treated SBM

| Main Effects | | | | | | | | | |
|---|---|---|---|---|---|---|---|---|---|
| | Biomass | | | | | | | | |
| | Addition | | | | | Treatment Means | | | |
| | Level¹ | | Metals | | | 25% | | 35% | |
| TREATMENT | 25% | 35% | 0 | 500 | Mean | 0 | 500 | 0 | 500 |
| Heated Soybean Meal | 66.8^{x} | 63.2^{y} | 61.8^{x} | 68.2^{y} | 65.0^{b} | 64.6^{a} | 69.0^{a} | 61.4^{a} | 67.4^{b} |
| SBM plus Brewer's yeast | 81.1^{x} | 77.3^{y} | 77.5 | 81.0 | 79.2^{d} | 78.8^{b} | 83.3^{c} | 76.0^{c} | 78.6^{c} |
| SBM plus Citric Acid Fermentation | 68.5 | 67.8 | 63.5^{x} | 72.8^{y} | 68.2^{c} | 62.6^{a} | 74.4^{b} | 64.5^{b} | 71.1^{b} |
| SBM plus Citric Acid Yeast Cream | 64.2^{x} | 58.5^{y} | 60.1 | 62.6 | 61.4^{a} | 63.8^{a} | 64.7^{a} | 56.4^{a} | 60.6^{a} |
| Mean | 70.2^{x} | 66.7^{y} | 65.7^{x} | 71.2^{y} | 67.8 | 67.4^{y} | 72.9^{z} | 64.0^{x} | 69.4^{y} |

¹Biomass added at a rate sufficient to contribute 25 or 35% added moisture.
^{a,b,c}Means within columns with different superscripts are different P < .05.
^{x,y,z}Means within row and treatment factor with different superscripts are different P < .05, P < .10 respectively.
*Metal addition at 500 ppm each of Zn, Mn and Fe.

### Example 11

### Combination of Metals and Plant Extracts

In this study the effect of the combination of concentrated plant extracts and metals on ammonia production during digestion of SBM was assessed. Ammonia is produced during rumen digestion of protein. Therefore, reduced ammonia levels are indicative of reduced rumen digestion of protein. The plant extracts consisted of saponins from the Yucca schidigera plant, whereas the metal consisted of zinc in the form of zinc sulfate.

In this study, four rumen canulated lactating Holstein cows were used in a fermentation study to evaluate the effect of four diets: 1) low soluble protein control diet (positive control), 2) high soluble protein control diet (negative control), 3) high soluble protein diet with plant extract, and 4) high soluble protein diet with plant extract and metal, using a 4×4 Latin square design experiment. The composition of the test diets is disclosed in Table 18: Test Feed Ingredient Composition.

TABLE 18
Test Feed Ingredient Composition

| Ingredient | Diet 1 | Diet 2 | Diet 3 | Diet 4 |
|---|---|---|---|---|
| Plant extract | | | 0.3% | 0.3% |
| Zinc sulfate | | | | 3.0% |
| Urea | | 6.7% | 6.7% | 6.7% |
| Blood meal | 6.7% | | | |
| Product 621 Cow Power¹ | 26.7% | 26.7% | 26.7% | 26.7% |
| Product 610 Moorbuff² | 33.3% | 33.3% | 33.3% | 33.3% |
| Linseed meal | 33.3% | 33.3% | 33.0% | 30.0% |

¹Composition (g/100 g): Monocalcium phosphate 42.6; Salt 30; Magnesium oxide 11.9; Calcium sulfate 6.3; trace minerals, molasses and petrolatum 9.2. Manufactured by ADM Alliance Nutrition, Inc., Quincy, IL.
²Composition (g/100 g): Calcium carbonate 33.8; Distillers dried grains 18.0; Sodium sesquicarbonate 16.8; Dry brewer's yeast 12.0; Magnesium oxide 5.0; Potassium chloride 5.0; Molasses, pelleting agent and petrolatum 9.4. Manufactured by ADM Alliance Nutrition, Inc., Quincy, IL.

Rumen samples were taken every 30 minutes for five hours post feeding. Samples were acidified with 1 mL of 7.2 N sulfuric acid, centrifuged and the liquid portion subjected to ammonia concentration analysis. The results were: Diet 1=7.18 mg/dl; Diet 2=8.8 mg/dl; Diet 3=8.32 mg/dl; and Diet 4=7.88 mg/dl. The ammonia levels for Diet 4 containing the plant extract and the metal was lower than the levels for Diets 3 (containing the plant extract alone) and Diet 2 (containing a high soluble protein diet).

### Example 12

### Fermentation Biomass

In this study the effect of the addition of a fermentation biomass on the RUP content of oilseed and oilseed meals was assessed. Addition of brewer's yeast was compared to the addition of soy hulls.

Samples of protein substrates were mixed for 3 minutes in a Hobart mixer with treatments (brewer's yeast or soy hulls, as appropriate) and 15% or 25% added water (vol/wt). The protein mixtures were then weighted into 8 inch by 8 inch glass dishes, covered with aluminum foil, and placed into a 105° C. oven for 4 hours. After 4 hours, the foil was removed and the samples weighed, transferred to a 50° C. oven, and dried to 12% moisture content as estimated by weight loss.

Brewer's yeast (“BY”) was added to the protein substrates and processed as indicated above. The first set of 5 samples was created by adding BY diluted 50:50 with distilled water. The 50% BY dilution was added at a rate of 15% moisture addition to the protein substrates. The BY samples were compared to protein substrates containing 5.5% soy hulls and 25% water (vol/wt) processed with moist heat as described above. Protein substrates consisted of SBM, canola meal (“CM”), whole soybeans, rapeseed meal (“RM”), and rapeseed.

The effects of the treatments are detailed in Tables 19-21. As indicated in Table 19, the BY treatment had a statistically significant greater effect in creating RUP across all protein substrates. The protein substrates themselves reacted to the processes differently. The processed SBM and whole soybeans had the most RUP in both the BY treatment and the soy hulls treatment, with the BY treatment having the most RUP in both the SBM and whole soybeans, with 84.2% and 83.2%, respectively. The soy hull treatment produced 73.7% RUP for soybeans and 67.5% RUP for SBM, indicating there may be an interaction of prior processing form and RUP potential.

TABLE 19
Rumen Undegradable Protein (RUP) content of Oilseeds/Oilseed Meals
as Affected by Additives Used during Moist-Heat Processing

| | | | | | | Stat. | |
|---|---|---|---|---|---|---|---|
| | RUN 1 | | RUN 2 | | RUP % | Sig. | Improvem't |
| SAMPLE DESCRIPTION | REP 1 | REP 2 | REP 1 | REP 2 | AVERAGE | Groups | BY − SH |
| SBM + Brewer's Yeast | 85.1 | 86.4 | 84.8 | 80.6 | 84.2 | A | 16.7 |
| SBM + Soy Hulls | 75.1 | 66.7 | 67.3 | 61 | 67.5 | A | |
| CM + Brewer's Yeast | 63.9 | 60.3 | 63.0 | 54.7 | 60.5 | B | 10.3 |
| CM + Soy Hulls | 55.9 | 48.5 | 51.9 | 44.4 | 50.2 | B | |
| Soybeans + Brewer's Yeast | 87.9 | 81.5 | 84.1 | 79.1 | 83.2 | A | 9.5 |
| Soybeans + Soy Hulls | 75.3 | 78.0 | 74.1 | 67.2 | 73.7 | A | |
| RM + Brewer's Yeast | 64.9 | 56.8 | 60.0 | 63.3 | 61.3 | B | 7.6 |
| RM + Soy Hulls | 56.1 | 58.2 | 49.9 | 50.5 | 53.7 | B | |
| Rapeseed + Brewer's Yeast | 58.5 | 48.0 | 42.4 | 42.4 | 47.8 | C | 2.6 |
| Rapeseed + Soy Hulls | 49.9 | 46.7 | 40.4 | 43.8 | 45.2 | C | |

Pairwise comparison with SNK aposteriori contrasts P < .05.
A, B, C Means with different letters are different (P < .05).

| | |
|---|---|
| SBM + Brewer's Yeast | Heated SBM, 15% water added from a 50:50 mix of brewer's |
| | yeast and water |
| SBM + Soy Hulls | Heated SBM, 5.5% soy hulls, 25% water |
| CM + Brewer's Yeast | Canola Meal, 15% water added from a 50:50 mix of brewer's |
| | yeast and water |
| CM + Soy Hulls | Canola Meal, 5.5% soy hulls, 25% water |
| Soybeans + Brewer's Yeast | Soy beans, 15% water added from a 50:50 mix of brewer's yeast and water |
| Soybeans + Soy Hulls | Soy beans, 5.5% soy hulls, 25% water |
| RM + Brewer's Yeast | RM, 15% water added from a 50:50 mix of brewer's yeast and water |
| RM + Soy Hulls | RM, 5.5% soy hulls, 25% water |
| Rapeseed + Brewer's Yeast | Rapeseed, 15% water added from a 50:50 mix of brewer's yeast and water |
| Rapeseed + Soy Hulls | Rapeseed, 5.5% soy hulls, 25% water |

The CM and RM responded similarly to BY and soy hulls with respect to RUP formation, with the BY treatment showing higher RUP than the soy hull treatment. CM showed 60.5% and 50.2% RUP for BY and soy hulls, respectively, and RM showed 61.3% and 53.7% RUP for BY and soy hulls, respectively. Rapeseed was the least responsive to the treatment, showing 47.8% and 45.2% RUP for BY and soy hulls, respectively.

Table 20 lists the average effects on RUP content for each protein substrate for both BY treatment and soy hull treatment, compared to the same protein substrate with no additions. For all substrates tested, BY showed a greater increase for RUP content compared to soy hulls or no additions. Table 21 compares the overall effect on RUP content of oilseed/oilseed meal from adding BY versus soy hulls during a moist-heat processing method.

TABLE 20
Average Effects on RUP Content (% of CP) of Adding Soy Hulls or
Brewer's Yeast to a Moist-Heat Processing Method

| | Treatment | | | | |
|---|---|---|---|---|---|
| | | | Increase, | Brew. | Increase, |
| Oilseed | None* | Soy Hulls | Units of RUP | Yeast | Units of RUP |
| Soybean Meal | 35.0 | 67.5 | 32.5 | 84.2 | 49.2 |
| Canola Meal | 30.0 | 50.2 | 20.2 | 60.5 | 30.5 |
| Soy Beans − Whole | 28.0 | 73.7 | 45.7 | 83.2 | 55.2 |
| Rapeseed Meal | 30.0 | 53.7 | 23.7 | 61.3 | 31.3 |
| Rapeseed | ˜28.0 | 45.2 | 17.2 | 47.8 | 19.8 |

*Literature value from Preston, R. L. 2004 Typical Composition of Feeds for Cattle and Sheep. Beef, May, Pgs. 20-30.

TABLE 21
Overall Effect on RUP of Oilseeds/Oilseed Meals of Adding
Brewer's Yeast or Soy Hulls to a Moist-Heat Processing Method

| | RUP, % of CP |
|---|---|
| Brewer's Yeast | 67.4^{A} |
| Soy Hulls | 58.1^{B} |

Pairwise comparison with SNK aposteriori contrasts P < .05.
^{A,B}Means with different letters are different (P < .05).

### Example 13

### Rate of RUP Formation with Brewer's Yeast Biomass

In this study, the rate of RUP formation under moist-heat treatment conditions with and without the addition of brewer's yeast biomass was assessed. The effect of moist-heat processing time was also examined.

Samples of SBM were treated to moist-heat according to the procedure of Example 12. Samples were removed from the moist-heat process after 1, 2, 3, 4, 5, 12, and 24 hours. The results on RUP content and other values are listed in Table 22.

TABLE 22
Effect of Cooking Time on RUP Content of Moist-Heat Treated Soybean Meal With or
Without Brewer's Yeast

| | Heated Soybean Meal, 25% Water | | | | | | | Brewer's Yeast | | | | | | | |
|---|---|---|---|---|---|---|---|---|---|---|---|---|---|---|---|
| Cooking Time, hrs | 1 | 2 | 3 | 4 | 5 | 12 | 24 | 1 | 2 | 3 | 4 | 5 | 12 | 24 | AminoPlus |
| Moisture, % | 12.00 | 12.20 | 10.70 | 12.90 | 12.80 | 12.20 | 8.50 | 13.10 | 12.60 | 13.70 | 13.60 | 14.20 | 14.30 | 9.80 | 12.40 |
| Protein, % | 47.50 | 47.70 | 47.20 | 47.20 | 47.30 | 48.00 | 50.60 | 47.50 | 47.70 | 46.80 | 47.70 | 48.20 | 48.30 | 50.80 | 45.70 |
| Lysine, % | | | | 2.85 | | 2.43 | 1.71 | 2.75 | 2.56 | | 2.33 | | 1.78 | | |
| SIP, % CP | 10.80 | 10.72 | 8.75 | 8.88 | 6.48 | 6.85 | 6.55 | 14.47 | 8.16 | 5.43 | 6.78 | 8.39 | 7.71 | 6.26 | 6.32 |
| Fermentable DM, % | 76.01 | 69.96 | 65.86 | 59.55 | 58.13 | 46.72 | 36.84 | 76.55 | 58.81 | 53.18 | 47.93 | 45.81 | 41.78 | 34.55 | 54.35 |
| RUP, % CP | 41.03 | 51.45 | 57.72 | 69.39 | 69.35 | 83.77 | 89.41 | 39.49 | 68.00 | 76.84 | 81.89 | 83.00 | 85.60 | 88.57 | 75.72 |
| Lysine, % (In situ residue) | 4.48 | 4.44 | 4.30 | 4.54 | 4.34 | 3.72 | 2.46 | 4.20 | 3.91 | 3.66 | 3.77 | 3.43 | 2.62 | 1.99 | 4.21 |
| RUP-Lys % | 1.07 | 1.33 | 1.47 | 1.84 | 1.82 | 1.98 | 1.55 | 0.98 | 1.61 | 1.71 | 1.96 | 1.86 | 1.53 | 1.30 | 1.92 |
| RUP-Lys % Lys | | | | 64.43 | | 81.56 | 90.87 | 35.81 | 62.92 | | 84.26 | | 85.69 | | |
| Digestible RUP, % RUP | 93.42 | 85.02 | 96.14 | 93.20 | 94.77 | 88.44 | | 86.36 | 98.30 | 96.85 | 80.09 | 79.51 | 33.74 | | |
| Digestible Lys, % | | | | 1.71 | | 1.75 | | 0.85 | 1.58 | | 1.57 | | 0.51 | | |
| Digestible Lys, % of Lys | | | | 60.05 | | 72.13 | | 30.93 | 61.85 | | 67.49 | | 28.91 | | |

Addition of BY to the SBM followed by moist-heat processing increased the rate of RUP formation. An RUP value of about 77% was reached after 3 hours of heating as compared with an RUP value of 58% for 3 hours of heating of SBM without BY addition. Brewer's yeast addition also decreased the lysine content at all time points.

### Example 14

### Effect of Yeast Source and Type on RUP Formation

In this study, the effect of the type and source of yeast on the RUP content of SBM was assessed. SBM composition with several different types of yeasts were examined and compared to SBM and SBM with soy hulls.

The SBM and SBM additive compositions were treated with moist-heat processing as set forth in Example 12. For each yeast sample, the sample was diluted to 8% dry matter (“DM”) content with distilled water and added to SBM to allow for the addition of 25% water. Each sample was equalized to a 2% DM addition to the SBM and compared to the standard addition of 25% water and 5.5% soy hull addition as positive controls.

The yeast samples examined were Sensient yeast cell wall (commercially available from Sensient Technologies Corp., Milwaukee, Wis.); Sensient yeast (commercially available from Sensient Technologies Corp., Milwaukee, Wis.); Lallemand Instant yeast (baker's yeast, commercially available from Lallemand Inc., Montreal, Quebec, Canada); Lesaffre cream yeast (commercially available from Lesaffre Yeast Corporation, Milwaukee, Wis.); ADM yeast cell mass (corn fiber fermentation, commercially available from Archer Daniels Midland, Decatur, Ill.); and brewer's yeast (obtained from the F.L. Emmert Co. of Cincinnati, Ohio). In addition, four types of Brewer's grains were tested: two from Anheuser Busch (Pennsylvania brewery and Ohio brewery), Miller's Brewing Co. (Ohio brewery), and Mad Anthony's Brewery (Ft. Wayne, Ind.). The neutral detergent fiber (“NDF”) content of each Brewer's grain was measured and the DM was adjusted to 8% and incorporated at a rate sufficient to deliver 25% of added water. A control sample containing 4.9 g corn stover, a level sufficient to deliver amount of NDF similar to the addition of the Brewer's grains, was also tested. For each treatment, samples were produced on two separate days. After production, the samples were assayed for RUP content. The RUP content results are presented in Table 23.

TABLE 23
Evaluation of Yeast Sources for Use in the Formation of
RUP

| | DMD | RUP |
|---|---|---|
| Soybean Meal | 56.65^{abcd} | 65.10^{ef} |
| SBM + Soy Hulls 55% | 59.30^{abc} | 74.40^{abcd} |
| SBM + Brewers Yeast − Emmert | 52.95^{cde} | 74.35^{abcd} |
| SBM + LaSaffre Cream Yeast | 45.40^{f} | 83.30^{a} |
| SBM + Lallemand Instant Yeast | 48.20^{ef} | 81.75^{a} |
| SBM + ADM Yeast Cell Mass − C1/C2 | 51.05^{def} | 79.35^{ab} |
| SBM + ADM Yeast Cell Mass − C3/C4 | 54.25^{bcde} | 75.55^{abc} |
| SBM + Sensient Yeast | 57.75^{abc} | 71.30^{bcde} |
| SBM + Sensient Yeast Cell Wall | 62.85^{a} | 64.05^{ef} |
| SBM + CitriStim | 60.55^{ab} | 65.40^{def} |
| SBM + Corn Stover 4.9 g | 59.05^{abc} | 68.40^{cdef} |
| SBM + Anheiser Busch Brewers Grains #1 | 61.85^{a} | 63.45^{ef} |
| SBM + Anheiser Busch Brewers Grains #2 | 61.55^{a} | 63.20^{ef} |
| SBM + Miller's Brewing Brewers Grains | 61.25^{a} | 61.80^{f} |
| SBM + Mad Anthony's Brewers Grains | 61.05^{a} | 63.15^{ef} |

^{a,b,c,d,e,f}Means within column with different superscript are different (P < .05)

The commercial Lallemand and LaSaffre yeast products were most effective, with slightly greater average RUP than the ADM yeast cell mass (corn fiber fermentation). The Sensient yeast and the brewer's yeast had similar results and were equivalent to the soy hull control's RUP content. Addition of brewer's grain showed no additional increase in RUP content compared to moist-heat treated SBM or the corn stover controls.

### Example 15

### Fermentation Biomass and Liquid Lysine

In this study the effect on RUP content of SBM or raw soybeans under AminoPlus processing conditions with brewer's yeast additive and additional 5% liquid lysine product was examined.

The protein substrate was either SBM or raw boybeans. Treatment #1 comprised the protein substrate and 5.5% soy hulls with 25% added water. Treatment #2 comprised the protein substrate with brewer's yeast and 15% added moisture. Treatment #3 comprised the protein substrate with brewer's yeast, 5% liquid lysine added prior to the dry down step (i.e., when the sample is dried at 50° C. to 12% moisture), and 15% added moisture. All samples were processed with the moist-heat processing described in Example 12, and assayed for RUP content, lysine content, and bypass lysine content. Duplicate samples were produced on two separate days. The results are summarized in Table 24 and the averages for the treatments presented in Table 25.

The brewer's yeast containing Treatments #2 and #3 created 17 more RUP units for SBM (83% vs. 66%) and 11 more units for raw beans (81.5% vs. 70%). The addition of liquid lysine did not improve RUP production in either SBM or raw beans. SBM processing did not affect total RUP creation across treatments compared to raw beans.

The digestible RUP's were similar across treatments for SBM (96.5%-98.4%). For the raw beans, the digestible RUP's were less than SBM for all treatments (75.9%-60.83%). The lysine levels were much higher for all treatments for SBM over the raw beans. The control (Trt. #1) had slightly more lysine for SBM meal compared to the brewer's yeast treatments (Trts. #2 & #3) but they were similar for the raw beans.

The SBM bypass lysine amounts were higher for the brewer's yeast treatments (76% vs. 58%) over the control. The numbers were similar for raw soybeans. The digestible bypass lysine values were very good for all treatments of SBM (96%-98.5%). These values were less for raw beans (58%-80%) and the control treatment #1 appeared to be greatest. The values for soluble intake lysine are highly variable for all treatments.

More RUP was created with the brewer's yeast treatments in both substrates (SBM and raw soybeans). The digestibility of the RUP was high for all treatments of SBM. Digestibility of RUP for raw soybeans was lower. The bypass lysine values for both substrates indicate that the brewer's yeast treatments may be protecting additional lysine through the RUP process. The digestible bypass lysine is excellent across all treatments for SBM. Digestible bypass lysine was lower for raw beans. For all analyses, there were more variations in the values for raw beans than there was for SBM.

TABLE 24
SUMMARY OF RESULTS

| | RUP | | | | | | | DIGESTIBLE RUP | | | | | | |
|---|---|---|---|---|---|---|---|---|---|---|---|---|---|---|
| | RUN 1 | | RUN 2 | | | | | RUN 1 | | RUN 2 | | | | |
| | REP | REP | REP | REP | | STD | | REP | REP | REP | REP | | STD | |
| TREATMENT | 1 | 2 | 1 | 2 | AVG | DEV | C.V. % | 1 | 2 | 1 | 2 | AVG | DEV | C.V. % |
| Heated SBM, 5.5% soy hulls, 25% water | 63.20 | 70.11 | 67.30 | 64.40 | 66.25 | 3.09 | 4.67 | 98.7 | 98.2 | 98.3 | 98.3 | 98.38 | 0.22 | 0.23 |
| Heated SBM, 15% water added from a 50:50 | 82.10 | 84.27 | 85.10 | 80.80 | 83.07 | 1.97 | 2.37 | 96.0 | 98.9 | 98.5 | 99.3 | 98.18 | 1.49 | 1.51 |
| mix of brewer's yeast and water (no 5% | | | | | | | | | | | | | | |
| liquid lysine added) | | | | | | | | | | | | | | |
| Heated SBM, 15% water added from a 50:50 | 84.30 | 85.86 | 83.00 | 80.40 | 83.39 | 2.31 | 2.77 | 93.4 | 96.1 | 98.3 | 98.0 | 96.45 | 2.25 | 2.34 |
| mix of brewer's yeast and water (5% liquid | | | | | | | | | | | | | | |
| lysine added) | | | | | | | | | | | | | | |
| Raw soy beans, 5.5% soy hulls, 25% water | 64.80 | 77.70 | 68.80 | 69.20 | 70.13 | 5.43 | 7.74 | 65.9 | 79.2 | 74.9 | 83.6 | 75.90 | 7.55 | 9.95 |
| Raw soy beans, 15% water added from a | 79.30 | 85.98 | 80.40 | 80.30 | 81.50 | 3.03 | 3.72 | 52.1 | 54.2 | 64.3 | 72.7 | 60.83 | 9.54 | 15.69 |
| 50:50 mix of brewer's yeast and water | | | | | | | | | | | | | | |
| (no 5% liquid lysine added) | | | | | | | | | | | | | | |
| Raw soy beans, 15% water added from a | 79.30 | 88.74 | 80.20 | 77.00 | 81.31 | 5.13 | 6.31 | 58.2 | 53.0 | 79.9 | 62.3 | 63.35 | 11.67 | 18.42 |
| 50:50 mix of brewer's yeast and | | | | | | | | | | | | | | |
| water (5% liquid lysine added) | | | | | | | | | | | | | | |
| | LYSINE | | | | | | | SI-LYSINE | | | | | | |
| | RUN 1 | | RUN 2 | | | | | RUN 1 | | RUN 2 | | | | |
| | REP | REP | REP | REP | | STD | | REP | REP | REP | REP | | STD | |
| TREATMENT | 1 | 2 | 1 | 2 | AVG | DEV | C.V. % | 1 | 2 | 1 | 2 | AVG | DEV | C.V. % |
| Heated SBM, 5.5% soy hulls, 25% water | 4.05 | 3.88 | | | 3.97 | 0.12 | 3.03 | 8.2 | 4.9 | 2.5 | 12.8 | 7.10 | 4.46 | 62.83 |
| Heated SBM, 15% water added from a 50:50 | 3.66 | 3.72 | | | 3.69 | 0.04 | 1.15 | 4.7 | 10.0 | 5.6 | — | 6.77 | 2.84 | 41.91 |
| mix of brewer's yeast and water (no 5% | | | | | | | | | | | | | | |
| liquid lysine added) | | | | | | | | | | | | | | |
| Heated SBM, 15% water added from a 50:50 | 3.73 | 3.77 | | | 3.75 | 0.03 | 0.75 | 5.9 | 9.8 | 15.7 | 5.3 | 9.18 | 4.79 | 52.16 |
| mix of brewer's yeast and water (5% liquid | | | | | | | | | | | | | | |
| lysine added) | | | | | | | | | | | | | | |
| Raw soy beans, 5.5% soy hulls, 25% water | 2.44 | 2.40 | | | 2.42 | 0.03 | 1.17 | 9.0 | — | 13.7 | — | 11.35 | 3.32 | 29.28 |
| Raw soy beans, 15% water added from a | 2.43 | 2.49 | | | 2.46 | 0.04 | 1.72 | 4.3 | — | 8.6 | 3.8 | 5.57 | 2.64 | 47.40 |
| 50:50 mix of brewer's yeast and | | | | | | | | | | | | | | |
| water (no 5% liquid lysine added) | | | | | | | | | | | | | | |
| Raw soy beans, 15% water added from a | 2.26 | 2.60 | | | 2.43 | 0.24 | 9.89 | 7.7 | 8.2 | 7.7 | 5.9 | 7.38 | 1.01 | 13.71 |
| 50:50 mix of brewer's yeast and | | | | | | | | | | | | | | |
| water (5% liquid lysine added) | | | | | | | | | | | | | | |
| | BYPASS LYSINE | | | | | | | DIGESTIBLE BYPASS LYSINE | | | | | | |
| | RUN 1 | | RUN 2 | | | | | RUN 1 | | RUN 2 | | | | |
| | REP | REP | REP | REP | | STD | | REP | REP | REP | REP | | STD | |
| TREATMENT | 1 | 2 | 1 | 2 | AVG | DEV | C.V. % | 1 | 2 | 1 | 2 | AVG | DEV | C.V. % |
| Heated SBM, 5.5% soy hulls, 25% water | 52.9 | 48.6 | 77.3 | 55.2 | 58.50 | 12.83 | 21.93 | 94.9 | 95.3 | 98.8 | 95.7 | 96.18 | 1.78 | 1.85 |
| Heated SBM, 15% water added from a 50:50 | 76.8 | 76.0 | 77.6 | — | 76.80 | 0.80 | 1.04 | 98.6 | 97.9 | 98.6 | 99.2 | 98.58 | 0.53 | 0.54 |
| mix of brewer's yeast and water (no 5% | | | | | | | | | | | | | | |
| liquid lysine added) | | | | | | | | | | | | | | |
| Heated SBM, 15% water added from a | 80.2 | 67.0 | 82.8 | 75.1 | 76.28 | 6.96 | 9.13 | 95.2 | 96.6 | 98.2 | 97.6 | 96.90 | 1.31 | 1.35 |
| 50:50 mix of brewer's yeast and | | | | | | | | | | | | | | |
| water (5% liquid lysine added) | | | | | | | | | | | | | | |
| Raw soy beans, 5.5% soy hulls, 25% water | 61.6 | 70.4 | 42.1 | 73.6 | 61.93 | 14.16 | 22.86 | 82.0 | 89.2 | 69.3 | 80.4 | 80.23 | 8.23 | 10.26 |
| Raw soy beans, 15% water added from a | 77.9 | 56.9 | 61.8 | 65.6 | 65.55 | 8.97 | 13.68 | 78.7 | 79.5 | 26.4 | 47.3 | 57.98 | 25.84 | 44.58 |
| 50:50 mix of brewer's yeast | | | | | | | | | | | | | | |
| and water (no 5% liquid lysine added) | | | | | | | | | | | | | | |
| Raw soy beans, 15% water added from a | 69.2 | 77.8 | 73.5 | 78.8 | 74.83 | 4.40 | 5.88 | 81.4 | 79.7 | 59.3 | 45.5 | 66.48 | 17.22 | 25.90 |
| 50:50 mix of brewer's yeast | | | | | | | | | | | | | | |
| and water (5% liquid lysine added) | | | | | | | | | | | | | | |

TABLE 25
Treatment Averages

| | | | | | | | | Raw Soy Beans | | | | | | | | |
|---|---|---|---|---|---|---|---|---|---|---|---|---|---|---|---|---|
| | | Soybean Meal | | | | | | | | 15% | | 15% added | | | | |
| | | | | | | 15% added | | | | added | | moisture | | | | |
| | | | | | | moisture | | 25% added | | moisture | | from | | | | |
| | | 25% added | | 15% added | | from brewer's | | moisture + | | from | | brewer's | | | | |
| | | moisture + 5.5% | | moisture from | | yeast + 5% | | 5.5% | | brewer's | | yeast + 5% | | | | |
| | | soy hulls | | brewer's yeast | | Lysine | | soy hulls | | yeast | | Lysine | | P Values | | |
| | Amino | Trt. #1 | | Trt. #2 | | Trt. #3 | | Trt. #1 | | Trt. #2 | | Trt. #3 | | | | Pair- |
| | Plus | | Std. | | Std. | | Std. | | Std. | | Std. | | Std. | | | Wise |
| | Std. | Mean | Dev. | Mean | Dev. | Mean | Dev. | Mean | Dev. | Mean | Dev. | Mean | Dev. | Subst | Trt | Compare¹ |
| Moisture | 12.40 | 13.45 | 0.57 | 13.03 | 0.66 | 13.83 | 0.74 | 13.48 | 0.85 | 11.30 | 0.54 | 12.15 | 0.21 | | | |
| Protein | 45.70 | 45.65 | 0.88 | 48.15 | 0.42 | 48.08 | 0.68 | 34.25 | 0.53 | 36.58 | 0.34 | 36.13 | 0.78 | | | |
| Sol. DM | 28.80 | 30.28 | 0.77 | 28.53 | 0.62 | 29.28 | 0.85 | 18.03 | 2.98 | 18.73 | 4.73 | 18.25 | 1.64 | | | |
| Ferm. DM | 53.60 | 62.60 | 2.80 | 49.33 | 1.23 | 50.93 | 3.82 | 49.68 | 3.82 | 44.83 | 7.41 | 42.65 | 1.76 | | | |
| SIP | 6.30 | 5.30 | 3.53 | 4.95 | 1.05 | 5.75 | 0.61 | 2.50 | 1.90 | 2.67 | 2.05 | 2.70 | 2.33 | 0.01 | 0.92 | A |
| RUP | 72.60 | 66.25 | 3.09 | 83.07 | 1.97 | 83.39 | 2.31 | 70.13 | 5.43 | 81.50 | 3.03 | 81.31 | 5.13 | 0.96 | <0.0001 | BC |
| Dig. RUP | 98.70 | 98.38 | 0.22 | 98.18 | 1.49 | 96.45 | 2.25 | 75.90 | 7.55 | 60.83 | 9.54 | 63.35 | 11.67 | <0.0001 | 0.07 | A |

¹Letter refers to P < .05.
A = Heated vs. Raw
B = Trt 1 vs. Trt 2
C = Trt 1 vs. Trt 3

Although the foregoing description has necessarily presented a limited number of embodiments of the invention, those of ordinary skill in the relevant art will appreciate that various changes in the components, details, materials, and process parameters of the examples that have been herein described and illustrated in order to explain the nature of the invention may be made by those skilled in the art, and all such modifications will remain within the principle and scope of the invention as expressed herein in the appended claims. It will also be appreciated by those skilled in the art that changes could be made to the embodiments described above without departing from the broad inventive concept thereof. It is understood, therefore, that this invention is not limited to the particular embodiments disclosed, but it is intended to cover modifications that are within the principle and scope of the invention, as defined by the claims.

We claim:

1. An animal feed composition comprising:
an ingredient selected from the group consisting of an isolated enzyme, an organic acid, a fermentation biomass of a eukaryotic cell origin, and combinations of any thereof; and
at least one proteinaceous feed ingredient,
wherein the ingredient and the at least one proteinaceous feed ingredient are treated with a moist heat treatment, and
wherein upon administration of the animal feed composition to a ruminant, an amount of protein passing through a rumen of the ruminant is increased as compared to an animal feed composition that does not include the ingredient administered to the ruminant.

2. The animal feed composition of claim 1, wherein the ingredient comprises a fermentation biomass selected from the group consisting of a yeast, a yeast cream, a yeast biomass, a lysine biomass, a lactic acid fermentation biomass, a citric acid presscake, an ethanol presscake, distiller's yeast, a brewer's yeast biomass, a baker's yeast biomass, and mixtures of any thereof.

3. The animal feed composition of claim 2, wherein the ingredient further comprises an organic acid selected from the group consisting of ascorbic acid, citric acid, aconitic acid, malic acid, fumaric acid, succinic acid, pyrocitric acid, lysine, salts of any thereof, and combinations of any thereof.

4. The animal feed composition of claim 2, wherein the ingredient further comprises a gluten protein selected from the group consisting of a corn gluten protein, a rice globulin protein, a wheat gluten protein, and mixtures of any thereof.

5. The animal feed composition of claim 1, wherein the at least one proteinaceous feed ingredient is selected from the group consisting of soybeans, soybean meal, corn, corn meal, linseed, linseed meal, cottonseed, cottonseed meal, rapeseed, rapeseed meal, sorghum protein, canola meal, and combinations of any thereof.

6. The animal feed composition of claim 1, further comprising a compound selected from the group consisting of, a divalent metal ion, a plant extract, and combinations of any thereof.

7. A method of feeding an animal comprising:
treating a fermentation biomass of a eukaryotic origin and at least one proteinaceous feed ingredient; and
feeding a ruminant an animal feed composition comprising the treated fermentation biomass and the at least one proteinaceous feed ingredient,
wherein an amount of protein passing through a rumen of the ruminant is increased upon administration of the animal feed composition to the ruminant, as compared to an animal feed composition that does not include the treated fermentation biomass and the treated at least one proteinaceous feed ingredient administered to the ruminant.

8. The method of claim 7, wherein treating the fermentation biomass and the least one proteinaceous feed ingredient comprises:
heating the fermentation biomass and the at least one proteinaceous feed ingredient at a moisture content of from 15% to 50%; and
drying the heated fermentation biomass and the at least one proteinaceous feed ingredient to 10% to 15% moisture.

9. The method of claim 7, wherein the fermentation biomass is selected from the group consisting of a yeast, a yeast cream, a yeast biomass, a lysine biomass, a lactic acid fermentation biomass, a citric acid presscake, an ethanol presscake, distiller's yeast, a brewer's yeast biomass, a baker's yeast biomass, and combinations of any thereof.

10. The method of claim 7, further comprising adding an ingredient selected from the groups consisting of an organic acid, a gluten protein, an isolated enzyme, a divalent metal ion, a plant extract, and combinations of any thereof to the animal feed composition.

11. The method of claim 7, wherein treating comprises:
mixing the fermentation biomass and water; and
combining the fermentation biomass and water with the at least one proteinaceous feed ingredient to 15% to 50% moisture.

12. The method of claim 7, wherein treating the composition comprises:
heating the fermentation biomass and the at least one proteinaceous feed ingredient at a temperature from 87° C. to 116° C. in 15% to 50% moisture for 0.10 hours to 5 hours; and
drying the fermentation biomass and the at least one proteinaceous feed ingredient to 10% to 15% moisture.

13. The method of claim 7, further comprising forming the animal feed composition into a form selected from the group consisting of a meal, a pellet, a block, a tub, a premix, an additive, a top-dress, and a liquid feed supplement.

14. The method of claim 7, wherein feeding a ruminant the animal feed composition comprises feeding the ruminant animal feed composition in an amount of 0.454 kg/head/day to 3.18 kg/head/day.

15. The method of claim 7, wherein the animal feed composition is in the form of a premix and wherein feeding a ruminant the animal feed composition comprises feeding the premix to the ruminant in an amount of 0.09 kg/head/day to 0.454 kg/head/day.

16. A process for producing a feed supplement comprising:
mixing a composition comprising a fermentation biomass of a eukaryotic origin and at least one proteinaceous feed ingredient;
treating the composition with moist heat; and
forming the composition into a form selected from the group consisting of a meal, a pellet, a block, a tub, a premix, an additive, and a liquid feed supplement.

17. The process of claim 16, wherein the fermentation biomass is selected from the group consisting of a yeast, a yeast cream, a yeast biomass, a lysine biomass, a lactic acid fermentation biomass, a citric acid presscake, an ethanol presscake, distiller's yeast, a brewer's yeast biomass, a baker's yeast biomass, and mixtures of any thereof.

18. The process of claim 16, further comprising mixing an ingredient with the composition, wherein the ingredient is selected from the group consisting of an organic acid, a gluten protein, an isolated enzyme, a divalent metal ion, a plant extract, and combinations of any thereof.

19. The process of claim 16, wherein mixing the composition comprises:
mixing the fermentation biomass and water; and
combining the fermentation biomass and water with the at least one proteinaceous feed ingredient to a moisture of 10% to 50%.

20. The process of claim 16, wherein treating the composition with moist heat comprises:
heating the composition at 87° C. to 116° C. and 10% to 50% moisture for 0.10 hours to 5 hours; and
drying the composition to 10% to 15% moisture.

21. The process of claim 16, wherein the composition is in the form of a meal or a pellet.

22. The process of claim 16, further comprising:
placing the composition in a container configured for shipping; and
associating indicia with the container, wherein the indicia is capable of directing a user on how to administer the composition to an animal.

23. An animal feed composition comprising:
a yeast fermentation biomass; and
at least one proteinaceous feed ingredient,
wherein the yeast fermentation biomass and the at least one proteinaceous feed ingredient have been treated, and
wherein upon administration of the animal feed composition to a ruminant, an amount of protein passing through a rumen of the ruminant is increased as compared to an animal feed composition that does not comprise a treated yeast fermentation biomass and at least one proteinaceous feed ingredient administered to the ruminant.

24. The animal feed composition of claim 23, wherein the yeast fermentation biomass and the at least one proteinaceous feed ingredient are treated with moist heat.

25. The animal feed composition of claim 23, wherein the yeast fermentation biomass is selected from the group consisting of a yeast presscake, a yeast cream, a citric acid biomass, an ethanol biomass, distiller's yeast, a brewer's yeast biomass, a baker's yeast biomass, and combinations of any thereof.

26. The animal feed composition of claim 23, further comprising an ingredient selected from the group consisting of an isolated enzyme, a gluten protein, a divalent metal ion, an organic acid, a plant extract, and combination of any thereof.
